(19) United States
(12) Patent Application Publication
(10) Pub. No.: US 2003/0232400 A1
(43) Pub. Date: Dec. 18, 2003

(54) Methods of screening subjects for expression of soluble receptors of vascular endothelial growth factor (VEGF) for use in managing treatment and determining prognostic outcome

(76) Inventors: Susan Radka, Arvada, CO (US); Roger Aitchison, Niwot, CO (US); Pamela Pavco, Lafayette, CO (US)

Correspondence Address: Lisa M. W. Hillman, Ph.D., McDonnell Boehnen Hulbert & Berghoff, 32nd Floor, 300 S. Wacker Drive, Chicago, IL, 60606, US

(21) Appl. No.: 10/438,493

(22) Filed: May 15, 2003

Related U.S. Application Data
(60) Provisional application No. 60/435,941, filed on Dec. 20, 2002.

(51) Int. Cl. G01N 33/574; G01N 33/53; G01N 33/537; G01N 33/543; A61K 48/00

(52) U.S. Cl. 435/7.23; 435/7.92; 514/44

(57) ABSTRACT

This invention relates generally to compounds and methods relating to detection of soluble VEGFR expression for generating prognostic criteria useful in establishing methods of treatment in cancer patients. In the method of the present invention, soluble VEGFR levels are utilized as a means by which cancer can be detected, efficacy of cancer therapies can be evaluated, prognosis of a subject can be predicted, and new anti-cancer therapies can be discovered.

This patent application claims the benefit of U.S. Ser. No. 60/435,941, filed Dec. 20, 2002, entitled “METHODS OF SCREENING SUBJECTS FOR EXPRESSION OF SOLUBLE RECEPTORS OF VEGF FOR USE IN MANAGING TREATMENT AND DETERMINING PROGNOSTIC OUTCOME.” This application is hereby incorporated by reference herein in its entirety including the drawings.

### FIELD OF THE INVENTION

This invention relates generally to compounds and methods relating to detection of soluble VEGF receptor expression for generating prognostic criteria useful in establishing methods of treatment in cancer patients. In the method of the present invention, soluble VEGF receptor levels are utilized as a means by which cancer can be detected, efficacy of cancer therapies can be evaluated, prognosis of a subject can be predicted, and new anti-cancer therapies can be discovered or evaluated.

### BACKGROUND OF THE INVENTION

VEGF, also referred to as vascular permeability factor (VPF) and vasculotropin, is a potent and highly specific mitogen of vascular endothelial cells (for a review see Ferrara, 1993 Trends Cardiovas. Med. 3, 244; Neufeld et al., 1994, Prog. Growth Factor Res. 5, 89). VEGF-induced neovascularization is implicated in various pathological conditions such as tumor angiogenesis, proliferative diabetic retinopathy, hypoxia-induced angiogenesis, rheumatoid arthritis, psoriasis, wound healing and others.

VEGF, an endothelial cell-specific mitogen, is a 34-45 kDa glycoprotein with a wide range of activities that include promotion of angiogenesis, enhancement of vascular-permeability and others. VEGF belongs to the platelet-derived growth factor (PDGF) family of growth factors with approximately 18% homology with the A and B chain of PDGF at the amino acid level. Additionally, VEGF contains the eight conserved cysteine residues common to all growth factors belonging to the PDGF family (Neufeld et al., supra). VEGF protein is believed to exist predominantly as disulfide-linked homodimers; monomers of VEGF have been shown to be inactive (Plouet et al., 1989 EMBO J. 8, 3801). Several other members of the VEGF family have been cloned including VEGF-B, -C, and -D. Placenta growth factor (PlGF) is also closely related to VEGF-A. VEGF-A, -B, -C, -D, and PlGF are all distantly related to platelet-derived growth factors-A and -B. Less is known about the function and regulation of VEGF-B, -C, and -D, but they do not seem to be regulated by the major pathways that regulate VEGF-A.

VEGF exerts its influence on vascular endothelial cells by binding to specific high-affinity cell surface receptors. Covalent cross-linking experiments with ¹²⁵I-labeled VEGF protein have led to the identification of three high molecular weight complexes of 225, 195 and 175 kDa presumed to be VEGF and VEGF receptor complexes (Vaisman et al., 1990 J. Biol. Chem. 265, 19461). Based on these studies VEGF-specific receptors of 180, 150 and 130 kDa molecular mass were predicted. In endothelial cells, receptors of 150 and 130 kDa have been identified. The VEGF receptors belong to the superfamily of receptor tyrosine kinases (RTKs) characterized by a conserved cytoplasmic catalytic kinase domain and a hydrophilic kinase sequence. The extracellular domains of the VEGF receptors consist of seven immunoglobulin-like domains that are thought to be involved in VEGF binding functions.

There are three receptors in the VEGF receptor family. They have the common properties of multiple IgG-like extracellular domains and tyrosine kinase activity. The enzyme domains of VEGF receptor 1 (VEGFR1, also known as Flt-1), VEGFR2 (also known as KDR or Flk-1), and VEGFR3 (also known as Flt-4) are divided by an inserted sequence. Endothelial cells also express additional VEGF receptors, including Neuropilin-1 and Neuropilin-2. VEGF-A binds to VEGFR1 and VEGFR2 and to Neuropilin-1 and Neuropilin-2. PlGF and VEGF-B bind VEGFR1 and Neuropilin-1. VEGF-C and -D bind VEGFR3 and VEGFR2. The VEGF-C/VEGFR3 pathway is important for lymphatic proliferation. VEGFR3 is specifically expressed on lymphatic endothelium. A soluble form of Flt-1 can be detected in peripheral blood and is a high affinity ligand for VEGF. Soluble Flt-1 can be used to antagonize VEGF function. VEGFR1 and VEGFR2 are upregulated on tumor and proliferating endothelium, partly by hypoxia and also in response to VEGF-A itself. VEGFR1 and VEGFR2 can interact with multiple downstream signaling pathways via proteins such as PLC-g, Ras, Shc, Nck, PKC and PI3-kinase. VEGFR1 is of higher affinity than VEGFR2 and mediates motility and vascular permeability. VEGFR2 appears to be necessary for proliferation.

Vascular endothelial growth factor receptor-1 (VEGFR1, also known as Flt-1) was originally discovered through the screening of a human placental cDNA library. It is a receptor tyrosine kinase (RTK) specific for the angiogenic factors VEGF (VEGF-A), P/GF, and VEGF-B. VEGFR1 is expressed in two forms via alternate splicing at the pre-mRNA level; a full-length, membrane bound receptor capable of transducing signal, and a truncated, soluble receptor (sVEGFR1) capable of sequestering ligand or dimerizing with full-length receptor and preventing signal transduction. Though VEGFR1 null mutations are lethal, deletions of the kinase domain are not, suggesting that the soluble form, or at least the extracellular domain, is all that is necessary for normal vascular development. Experimental and clinical administrations of sVEGFR1 have been employed successfully in the prevention of neovasculogenesis and tumor growth. The serum/plasma level of sVEGFR1 may vary by pathology. The human VEGFR1 gene produces two major transcripts of 3.0 and 2.4 kb, corresponding to the full-length receptor and soluble receptor, respectively. Full length VEGFR1 is an approximately 180 kDa glycoprotein featuring seven extracellular immunoglobulin (Ig)-like domains, a membrane-spanning region, and an intracellular tyrosine kinase domain containing a kinase insert sequence. The truncated sVEGFR1 consists of only the first six extracellular Ig-like domains. Ligand binding takes place within the first three N-terminal Ig-like domains while the fourth Ig-like domain is responsible for receptor dimerization, which is a prerequisite for activation through trans-phosphorylation. In addition to homodimers, VEGFR1 can form active heterodimers with VEGFR2. The soluble form of VEGFR1 forms inactive heterodimers with VEGFR2. VEGF, VEGFR1, and VEGFR2 represent a regulatory system essential for both normal and pathological angiogenesis. Although VEGFR1 is expressed in significant levels on monocyte/macrophage lineages, expression of both VEGF receptors is primarily restricted to endothelial cells. VEGF, FGF basic, PECAM-1, cell-cell contact, and hypoxia are all reported to increase VEGF receptor expression. Peripheral blood monocyte expression of VEGFR1 is up-regulated in response to monocyte activation. High levels of sVEGFR1 reportedly occur in the plasma during pregnancy and in patients with essential hypertension. Significantly lower levels have been observed in the plasma of patients with cardiovascular disease and in smokers. The exact role of VEGFR1 in the angiogenic process has been disputed. VEGFR1 has a greater affinity for VEGF than VEGFR2, yet VEGFR2 is phosphorylated approximately 10-fold more efficiently upon ligand binding. VEGF stimulates chemotaxis and proliferation of VEGFR2-transfected, but not VEGFR1-transfected, human umbilical vein and porcine aortic endothelial cells. Knockout mice missing either receptor usually die by day 10. Absence of VEGFR2 results in an undeveloped vasculature and few mature endothelial cells while absence of VEGFR1 results in a preponderance of endothelial cells coalesced into disorganized tubules. Mice lacking only the tyrosine kinase domain of VEGFR1, however, survive with near normal vasculature. These observations support the conclusion that VEGFR1 functions to limit VEGF/VEGF R2 mediated angiogenesis with the intact receptor acting as a decoy and the soluble form creating inert receptors by dimerization with VEGFR2 or sequestering free ligand. There is evidence, however, that the tyrosine kinase domain of VEGFR1 does play an angiogenic role. VEGFR1/R2 heterodimers can transduce signal. VEGF-induced nitric oxide (NO) release appears to be mediated by VEGFR1, and this NO release in turn acts as a molecular switch, inhibiting VEGFR2-mediated proliferation and affecting endothelial cell redifferentiation into capillary like structures. Additionally, VEGF and P/GF mediated migration of monocytes/macrophages and production of tissue factor appears to be mediated by VEGFR1.

VEGF expression has been associated with several pathological states such as tumor angiogenesis, various cancers, several forms of blindness, rheumatoid arthritis, psoriasis, endometriosis and others. In addition, a number of studies have demonstrated that VEGF is both necessary and sufficient for neovascularization. Takashita et al., 1995 J. Clin. Invest. 93, 662, demonstrated that a single injection of VEGF augmented collateral vessel development in a rabbit model of ischemia. VEGF also can induce neovascularization when injected into the cornea. Expression of the VEGF gene in CHO cells is sufficient to confer tumorigenic potential to the cells. Kim et al., supra and Millauer et al., supra used monoclonal antibodies against VEGF or a dominant negative form of VEGFR2 receptor to inhibit tumor-induced neovascularization.

Kumar et al., 2002, Anticancer Research, 22, 1877-1880, describe an ELISA method to assay soluble VEGFR1 in human sera in colorectal and breast cancer patients compared to healthy volunteers. No soluble VEGFR1 was detected in the sera of the healthy volunteers, whereas soluble VEGFR1 was detected in the sera of patients with colorectal and breast cancer, however, no correlation was made between the soluble VEGFR1 and the stage of cancer, or between levels of soluble VEGFR1 in serum and membrane bound VEGFR1. Serum VEGFR1 was shown to be significantly reduced in postoperative patients with detectable preoperative levels of VEGFR1. In addtion, no preoperative serum negative for VEGFR1 became positive postoperatively (Kumar et al., supra). No prognostic significance was assigned to this data.

As such, there currently exists a need for assay methodologies that utilize VEGF receptors as biomarkers for malignancy, prognostic outcome, and treatment strategy in subjects. Such methods will identify subjects amenable to treatment with compounds that inhibit or down regulate expression and/or activity of VEGFR and can also serve to monitor existing treatments and/or new treatments in such subjects. In addition, methods of analysis of VEGFR in subjects will serve as a prognostic indicator of disease progression and/or outcome in response to various treatment strategies.

### SUMMARY OF THE INVENTION

The present invention relates to compounds and methods useful for the detection and assay of soluble VEGFR as a biological marker of cancer or susceptability to cancer in a subject or sample. In particular, the invention relates to methods of determining optimum treatment strategies for subjects expressing soluble VEGFR as a biomarker for cancer. Such methods will identify subjects amenable to treatment with compounds that inhibit or down regulate expression and/or activity of VEGFR (soluble and membrane bound) and/or VEGF and can also serve to monitor existing treatments and/or new treatments in such subjects. In addition, methods of analysis of VEGFR in subjects will serve as a prognostic indicator of disease progression and/or outcome in response to various treatment strategies.

In one embodiment, the invention features a method, comprising: (a) assaying the level of soluble VEGFR in a sample; and (b) correlating this level with the level of membrane bound VEGFR in the subject from which the sample is derived. In another embodiment, the sample can comprise a blood, serum, urine, or tissue sample from a subject, for example a cancer subject. In another embodiment, the assay can comprise ELISA or an equivalent assay for detecting soluble VEGFR. In yet another embodiment, the determination utilizes a standard curve of soluble VEGFR levels compared to membrane bound VEGFR levels. In another embodiment, the subject is a cancer subject, wherein the cancer is, for example, colorectal cancer, breast cancer, and/or non-small cell lung cancer (NSLC).

In another embodiment, the invention features a method for identifying cancer in subjects having elevated levels of membrane bound VEGFR and/or VEGF, comprising: (a) assaying the level of soluble VEGFR (e.g. VEGFR1) in a sample from the subject; (b) determining the level of membrane bound VEGFR and/or VEGF in the subject, and (c) determining whether the subject has cancer based upon the level of soluble or membrane bound VEGFR and/or VEGF in the subject. In another embodiment, the determination utilizes a standard curve of soluble or membrane bound VEGFR and/or VEGF levels compared to observed cancer phenotypes associated with soluble or membrane bound VEGFR and/or VEGF levels, for example as determined by a standard curve analysis of cancer phenotypes and non-cancer phenotypes.

In another embodiment, the invention features a method for determining efficacy of a cancer treatment in a subject, comprising: (a) assaying the level of soluble VEGFR in a sample from the subject prior to treatment; (b) assaying the level of soluble VEGFR in a sample from the subject during or after treatment; and (c) determining the efficacy of the treatment by comparing the level of soluble VEGFR in (a) compared to (b) under conditions suitable for the determination. For example, a level of soluble VEGFR that is lower in (b) compared to (a) indicates efficacy for a treatment, whereas a level of soluble VEGFR that is equal or higher in (b) compared to (a) indicates little or no efficacy for a treatment. In another embodiment, the determination utilizes a standard curve of soluble VEGFR levels compared to observed cancer phenotypes associated with certain soluble VEGFR levels, for example as determined by a standard curve compared to other cancer phenotypes or non-cancer phenotypes.

In another embodiment, the invention features a method for predicting prognostic outcome of a cancer subject, comprising: (a) assaying the level of soluble VEGFR in a sample from the subject; and (b) using the level of soluble VEGFR in the subject under conditions suitable to predict prognostic outcome of the subject. For example, a level of soluble VEGFR that is zero or very low indicates a good prognosis, whereas a level of VEGFR that is elevated indicates a poor prognosis. In another embodiment, the prediction utilizes a standard curve of soluble VEGFR levels compared to observed cancer phenotypes associated with certain soluble VEGFR levels, for example as determined by a standard curve compared to other cancer phenotypes or non-cancer phenotypes.

In another embodiment, the invention features a method for predicting prognostic outcome of a subject undergoing cancer treatment, comprising: (a) assaying the level of soluble VEGFR in a sample from the subject prior to treatment; (b) assaying the level of soluble VEGFR in a sample from the subject during or after treatment; and (c) predicting the prognostic outcome of the subject undergoing treatment by comparing the level of soluble VEGFR in (a) compared to (b) under conditions suitable for the prediction. For example, a level of soluble VEGFR that is lower in (b) compared to (a) indicates good prognostic outcome, whereas a level of soluble VEGFR that is equal or statistically unchanged in (b) compared to (a) indicates stable prognostic outcome, and a level of soluble VEGFR that is higher in (b) compared to (a) indicates poor prognostic outcome. In another embodiment, the prediction utilizes a standard curve of soluble VEGFR levels compared to observed cancer phenotypes associated with certain soluble VEGFR levels, for example as determined by a standard curve compared to other cancer phenotypes or non-cancer phenotypes.

In another embodiment, the invention features a method for evaluating the efficacy of an anti-cancer treatment, comprising: (a) assaying the level of soluble VEGFR in a sample from a cancer subject prior to treatment; (b) assaying the level of soluble VEGFR in a sample from the subject during or after treatment; and (c) determining the efficacy of the treatment by comparing the level of soluble VEGFR in (a) compared to (b) under conditions suitable for the determination. For example, a level of soluble VEGFR that is lower in (b) compared to (a) indicates good efficacy, whereas a level of soluble VEGFR that is statistically unchanged or greater in (b) compared to (a) indicates poor efficacy. In another embodiment, the determination of efficacy utilizes a standard curve of soluble VEGFR levels compared to observed cancer phenotypes associated with certain soluble VEGFR levels, for example as determined by a standard curve compared to other cancer phenotypes or non-cancer phenotypes.

In another embodiment, the invention features a method for evaluating the efficacy of an anti-angiogenic treatment, comprising: (a) assaying the level of soluble VEGFR in a sample from a cancer subject prior to treatment; (b) assaying the level of soluble VEGFR in a sample from the subject during or after treatment; and (c) determining the efficacy of the treatment by comparing the level of soluble VEGFR in (a) compared to (b) under conditions suitable for the determination. For example, a level of soluble VEGFR that is lower in (b) compared to (a) indicates good efficacy, whereas a level of soluble VEGFR that is statistically unchanged or greater in (b) compared to (a) indicates poor efficacy. In another embodiment, the determination of efficacy utilizes a standard curve of soluble VEGFR levels compared to observed cancer phenotypes associated with certain soluble VEGFR levels, for example as determined by a standard curve compared to other angiogenic phenotypes or non-angiogenic phenotypes.

In another embodiment, the invention features a method for evaluating the efficacy of a compound used to inhibit expression or activity of VEGF, comprising: (a) assaying the level of soluble VEGFR in a sample from a cancer subject prior to administration of the compound; (b) assaying the level of soluble VEGFR in a sample from the subject during or after administration; and (c) determining the efficacy of the compound by comparing the level of soluble VEGFR in (a) compared to (b) under conditions suitable for the determination. For example, a level of soluble VEGFR that is lower in (b) compared to (a) indicates good efficacy, whereas a level of soluble VEGFR that is statistically unchanged or greater in (b) compared to (a) indicates poor efficacy. In another embodiment, the determination of efficacy utilizes a standard curve of soluble VEGFR levels compared to observed VEGF levels, for example as determined by standard curve analysis.

In another embodiment, the invention features a method for evaluating the efficacy of a compound used to inhibit expression or activity of VEGFR, comprising: (a) assaying the level of soluble VEGFR in a sample from a cancer subject prior to administration of the compound; (b) assaying the level of soluble VEGFR in a sample from the subject during or after administration; and (c) determining the efficacy of the compound by comparing the level of soluble VEGFR in (a) compared to (b) under conditions suitable for the determination. For example, a level of soluble VEGFR that is lower in (b) compared to (a) indicates good efficacy, whereas a level of soluble VEGFR that is statistically unchanged or greater in (b) compared to (a) indicates poor efficacy. In another embodiment, the determination of efficacy utilizes a standard curve of soluble VEGFR levels compared to observed membrane bound VEGFR levels, for example as determined by standard curve analysis.

In one embodiment, the invention features a method of treating a subject comprsing: (a) assaying the level of soluble VEGFR in a sample from a subject; (b) treating the subject under conditions suitable for down regulating the level of soluble VEGFR and/or VEGF in the subject, and (c) monitoring the level of soluble VEGFR in the subject during or following treatment. In another embodiment, the sample can comprise a blood, serum, urine, or tissue sample. In another embodiment, the assay can comprise ELISA or an equivalent assay for detecting soluble VEGFR. In yet another embodiment, the assay utilizes a nucleic acid sensor molecule or aptamer. In another embodiment, the subject is a cancer subject, wherein the cancer is, for example, colorectal cancer, breast cancer, and/or non-small cell lung cancer (NSLC).

In one embodiment, a sample of the invention can comprise a blood, serum, urine, or tissue sample from a subject. In another embodiment, an assay of the invention can comprise ELISA or an equivalent assay for detecting soluble VEGFR in a sample. In yet another embodiment, a cancer contemplated by the invention is, for example, colorectal cancer, lung cancer (e.g NSLC), renal cancer, breast cancer and/or melanoma.

In one embodiment, assaying the level of soluble VEGFR in a sample from a subject comprises: (a) contacting the sample with pre-mixed capture reagents immobilized on a solid support under conditions suitable for any VEGFR in the sample to bind to the capture reagents; (b) separating the immobilized capture reagents and any bound VEGFR from the rest of the sample; (c) contacting the immobilized capture reagents and any bound VEGFR with a detatable antibody that binds to soluble VEGFR; and (d) measuring the level of soluble VEGFR bound to the capture reagents using a detection means for the detectable antibody. In another embodiment, the capture reagents comprise polyclonal or monoclonal antibodies against soluble human VEGFR. In another embodiment, the capture reagents comprise aptamers having binding affinity for soluble human VEGFR. In yet another embodiment, a detection means of the invention comprises a chromogenic, fluorescent, chemiluminescent, radioactive, or enzymatic detection system as is known in the art. Suitable enzymatic detection systems can include, for example, luciferase, horseradish peroxidase, and alkaline phosphatase.

In one embodiment, assaying the level of soluble VEGFR in a sample from a subject comprises: (a) contacting the sample with a nucleic acid sensor molecule and reporter molecule (see for example Usman et al., U.S. Ser. Nos. 10/056,761 and 09/877,526; George et al., U.S. Pat. Nos. 5,834,186 and 5,741,679 and Shih et al. U.S. Pat. No. 5,589,332); (b) measuring the level of signal generated by the reporter molecule in reponse to interaction of the nucleic acid sensor molecule and any soluble VEGFR in the sample; and (c) assaying the level of soluble VEGFR in the sample as determined by the level of signal generated in (b). In yet another embodiment, the reporter molecule comprises a detection means selected from the group consisting of a chromogenic, fluorescent, chemiluminescent, radioactive, or enzymatic detection system as is known in the art. Suitable enzymatic detection systems can include, for example, luciferase, horseradish peroxidase, and alkaline phosphatase.

In one embodiment, the invention features a nucleic acid sensor molecule that is used to assay the presence of soluble VEGFR in a system or sample, such as a biological system or sample, which is indicative of cancer. Non-limiting examples of cancers and cancerous conditions contemplated by the invention such as tumor angiogenesis and cancer, including but not limited to breast cancer, lung cancer (including non-small cell lung carcinoma), prostate cancer, colorectal cancer, brain cancer, esophageal cancer, bladder cancer, pancreatic cancer, cervical cancer, head and neck cancer, skin cancers, nasopharyngeal carcinoma, liposarcoma, epithelial carcinoma, renal cell carcinoma, gallbladder adeno carcinoma, parotid adenocarcinoma, ovarian cancer, melanoma, lymphoma, glioma, endometrial sarcoma, and multidrug resistant cancers.

In one embodiment, the invention features a nucleic acid sensor molecule having specificity for soluble VEGFR. In another embodiment, the invention features a nucleic acid sensor molecule having specificity for a conserved epitope of soluble VEGFR.

In one embodiment, the invention features a nucleic acid aptamer molecule that is used to assay the presence of soluble VEGFR in a system or sample, such as a biological system or sample, which is indicative of cancer or a cancerous condition described herein. In another embodiment, the invention features a nucleic acid aptamer molecule having specificity for a conserved epitope of soluble VEGFR.

The invention further includes detection methods, for example ELISA, cross-ligation, and gel-electrophoresis/gel-shift, using the antibodies, nucleic acid sensor molecules and nucleic acid aptamer molecules of the invention. In one embodiment, the invention provides methods for the detection of soluble VEGFR as a biological marker for cancer in a subject, including preclinical detection and/or preclinical screening of subjects having cancer.

In another embodiment, the invention features a non-invasive diagnostic assay for detecting the presence of a cancer or cancerous condition in a subject based on screening biological samples derived from a subject for levels of soluble VEGFR as a practical method for early detection of cancer.

In another embodiment, the invention features a non-invasive diagnostic assay for determining prognostic outcome of a subject having cancer, based on screening biological samples derived from a subject for levels of soluble VEGFR as a practical method for staging disease or predicting prognostic outcome.

The present invention also features methods for isolating nucleic acid molecules of the invention that are used in combination with the methods of detection and analysis of the invention. In one embodiment, the invention features a method for identifying nucleic acid aptamers of the invention having binding affinity for soluble VEGFR, comprising: (a) generating a randomized pool of oligonucleotides; (b) combining the oligonucleotides from (a) with soluble VEGFR under conditions suitable to allow at least one oligonucleotide in the pool to bind to soluble VEGFR; (c) partitioning oligonucleotide sequences (ligands) that bind to soluble VEGFR from unbound oligonucleotide sequences; (d) amplifying the oligonucleotide sequences isolated from (c) that bind to soluble VEGFR; (e) combining the oligonucleotides from (d) with soluble VEGFR under conditions suitable to allow at least one oligonucleotide to bind to soluble VEGFR; and (f) repeating steps (c), (d), and (e) under conditions suitable for isolating one or more nucleic acid molecules having binding affinity to soluble VEGFR. In another embodiment, step (d) is optionally carried out under conditions suitable for introducing some degree of mutation into the sequences in step (d).

In one embodiment, the invention features a method for generating nucleic acid sensor molecules capable of detecting the presence of soluble VEGFR in a system, comprising: (a) coupling a nucleic acid aptamer of the invention to an enzymatic nucleic acid molecule via a randomized nucleic acid sequence; (b) combining the oligonucleotides from (a) with soluble VEGFR, in vitro, under conditions suitable to allow target binding mediated catalysis of the enzymatic nucleic acid molecule; (c) isolating oligonucleotide sequences from (b) that possess catalytic activity by removing inactive oligonucleotide sequences; (d) amplifying the oligonucleotide sequences isolated from (c); and (e) repeating steps (c) and (d) under conditions suitable for isolating one or more nucleic acid sensor molecules having catalytic activity in the presence of soluble VEGFR. In one embodiment, the method comprises an additional step following (a), wherein sequences from (a) that possess catalytic activity in the absence of soluble VEGFR are removed. In one embodiment, step (d) is optionally carried out under conditions suitable for introducing some degree of mutation into the sequences. In another embodiment, the random region of (a) comprises a single stranded sequence. In yet another embodiment, the randomized nucleic acid sequence of (a) comprises a double stranded stem or stem loop structure.

In another embodiment, the invention features a method for generating nucleic acid sensor molecules capable of detecting the presence of soluble VEGFR in a system, comprising: (a) generating a pool of nucleic acid sequences having an enzymatic nucleic acid domain and a soluble VEGFR binding domain comprising one or more random regions of nucleotides; (b) combining the oligonucleotides from (a) with soluble VEGFR, in vitro, under conditions suitable to allow soluble VEGFR binding mediated catalysis of the enzymatic nucleic acid molecule; (c) isolating oligonucleotide sequences from (b) that possess catalytic activity by removing inactive oligonucleotide sequences; (d) amplifying the oligonucleotide sequences isolated from (c); and (e) repeating steps (c) and (d) under conditions suitable for isolating one or more nucleic acid sensor molecules having catalytic activity in the presence of soluble VEGFR. In one embodiment, the method comprises an additional step following (a), wherein nucleic acid sequences that possess catalytic activity in the absence of soluble VEGFR are removed. In another embodiment, step (d) is optionally carried out under conditions suitable for introducing some degree of mutation into the sequences. In another embodiment, a random region of (a) comprises a single stranded sequence. In yet another embodiment, a random region of (a) comprises a double stranded stem or stem loop structure.

In another embodiment, the invention features a method for generating nucleic acid sensor molecules capable of detecting the presence of soluble VEGFR in a system, comprising: (a) generating a pool of random enzymatic nucleic acid sequences; (b) combining the oligonucleotides from (a) with soluble VEGFR, in vitro, under conditions suitable to allow soluble VEGFR mediated catalysis of the enzymatic nucleic acid molecule; (c) isolating oligonucleotide sequences from (b) that possess catalytic activity by removing inactive oligonucleotide sequences; (d) amplifying the oligonucleotide sequences isolated from (c); and (e) repeating steps (c) and (d) under conditions suitable for isolating one or more nucleic acid sensor molecules having catalytic activity in the presence of soluble VEGFR. In one embodiment, the method comprises an additional step following (a), wherein enzymatic nucleic acid sequences that possess catalytic activity in the absence of soluble VEGFR are removed. In another embodiment, step (d) is optionally carried out under conditions suitable for introducing some degree of mutation into the sequences.

In one embodiment, each of the above described methods of selecting nucleic acid sensor molecules of the invention utilize cis cleavage of a reporter molecule that comprises a fixed nucleotide sequence for purposes of selection. In yet another embodiment, the methods of selecting nucleic acid sensor molecules of the invention utilize trans cleavage of a reporter molecule having a fixed sequence for purposes of selection.

In one embodiment, methods of the invention are applied to generate nucleic acid sensor molecules that are inactive in the presence of soluble VEGFR, for example, by selecting nucleic acid sensor molecules whose activity is inhibited in the presence of soluble VEGFR.

The random pool of oligonucleotides in the above methods can comprise DNA and/or RNA, with or without chemically modified nucleotides. When chemically modified nucleotides are used in the method, such modifications can be chosen such that a non-discriminatory polymerase will incorporate the chemically modified nucleotide into the oligonucleotide sequence when generated or amplified. Non-limiting examples of chemically modified nucleoside triphosphates (NTPs) that can be used in the method of the invention include 2′-deoxy-2′-fluoro, 2′-deoxy-2′-amino, 2′-O-alkyl, and 2′-O-methyl NTPs as well as various base modified NTPs, such as C5-modified pyrimidines, 2,6-diaminopurine, and inosine. The oligonucleotides used in the method can be of fixed or variable length.

In one embodiment, the method for identifying nucleic acid acids of the invention comprises attaching soluble VEGFR to a solid matrix, such as beads, microtiter plate wells, membranes, chip surfaces, or other solid matrices known in the art. In such a system, soluble VEGFR can be attached to the solid matrix either covalently or non-covalently. In another embodiment, the antibody, oligonucleotide or nucleic acid used in a method of the invention can be labeled, either directly or non-directly, for example, with a radioactive label, absorption label such as biotin, or a fluorescent label such as fluorescein or rhodamine.

In another embodiment, the method of identifying nucleic acids of the invention comprises a solution based method, where nucleic acid molecules having specificity for the target molecule or activity/inactivity in the presence of the target molecule can be isoloated by known methods, such as gel shift assays. In another embodiment, the oligonucleotide or nucleic acid used in a method of the invention can be labeled, either directly or non-directly, for example, with a radioactive label, absorption label such as biotin, or a fluorescent label such as fluorescein or rhodamine.

The present invention contemplates detection of soluble VEGFR protein, polypeptide, and/or peptide target molecules. For example, a method of the invention can be used on either whole proteins or peptides, for example, digested/fragmented protein products (see for example McSwiggen et al., U.S. Ser. No. 60/343,385, filed Oct. 19, 2001).

In another embodiment, the soluble VEGFR protein or peptide of the invention is a recombinant protein or peptide. In yet another embodiment, the soluble VEGFR protein or peptide of the invention is a synthetic protein or peptide.

In another embodiment, the invention features a kit comprising a nucleic acid sensor molecule, aptamer, or antibody of the invention. The kit of the invention can further include any additional reagents, reporter molecules, buffers, excipients, containers and/or devices as required described herein or known in the art, to practice a method of the invention.

In one embodiment, detection and/or quantitation of the presence of soluble VEGFR in the above inventive methods can be accomplished using a reporter molecule. The reporter molecule can be attached to the inventive enzymatic nucleic acid molecule and/or aptamer or can be free in the sample. In one embodiment, the reporter molecule of the instant invention comprises a detectable label selected from the group consisting of chromogenic substrate, fluorescent labels, chemiluminescent labels, and radioactive labels and enzymes. Suitable enzymes include, for example, luciferase, horseradish peroxidase, and alkaline phosphatase.

In another embodiment, the reporter molecule of the instant invention is immobilized on a solid support. Suitable solid supports include silicon-based chips, silicon-based beads, controlled pore glass, polystyrene, cross-linked polystyrene, nitrocellulose, biotin, plastics, metals and polyethylene films.

In one embodiment, the invention features an array of nucleic acid sensor molecules and/or aptamers comprising a predetermined number of nucleic acid sensor molecules and/or aptamers of the invention. In one embodiment, a nucleic acid sensor molecule or aptamer of the instant invention is attached to a solid surface. Preferably, the surface of the instant invention comprises silicon-based chips, silicon-based beads, controlled pore glass, polystyrene, cross-linked polystyrene, nitrocellulose, biotin, plastics, metals and/or polyethylene films.

In one embodiment, any of the inventive methods is carried out more than once.

### BRIEF DESCRIPTION OF THE DRAWINGS

FIG. 1 shows a non-limiting diagrammatic example of a method of screening subjects for expression of soluble VEGFR. The screening data can be used in various methods, for example to determine membrane bound VEGFR, predict prognostic outcome, determine therapeutic efficacy, or provide early detection of a cancer phenotype in a subject.

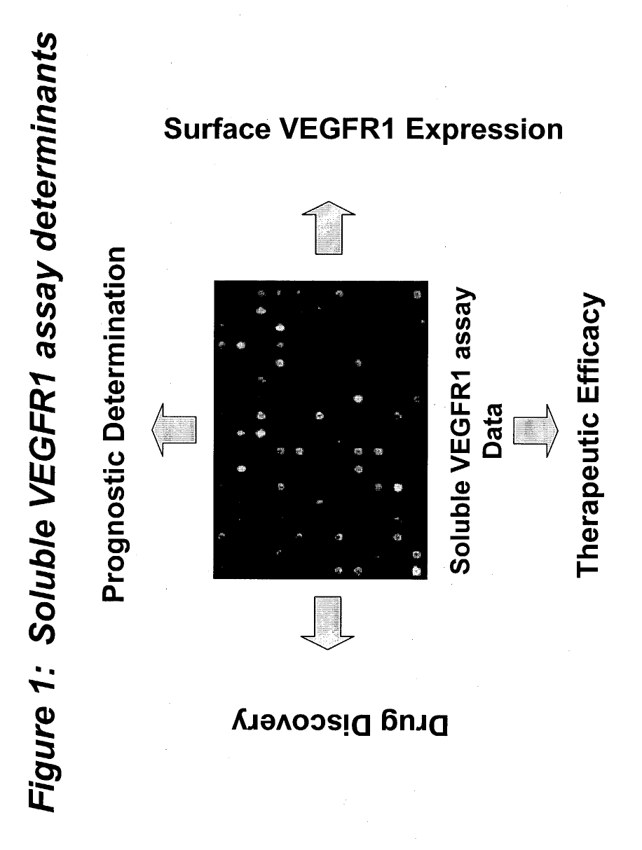

FIG. 2 shows a non-limiting diagrammatic example of a method for generating nucleic acid sensor molecules of the invention from a completely random pool of nucleic acid sequences (but having fixed binding arm sequences for interaction with a reporter molecule). The method comprises: (1) generating a random pool of nucleic acid sequences, (2) discarding any active sequences that have catalytic activity in the absense of target, (3) adding target soluble VEGFR to the pool, (4) discarding molecules that are inactive in the presence of the target, (5) amplification to enrich nucleic acid sensor sequences, and (6) repeating the process of (1-5) to increase nucleic acid sensor sensitivity and catalytic activity.

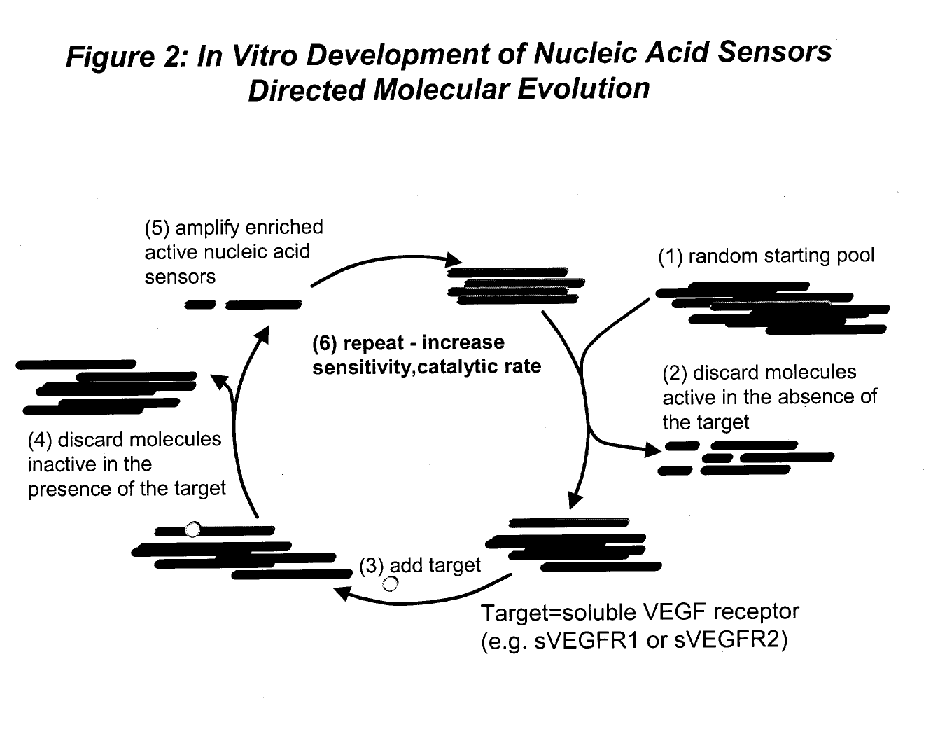

FIG. 3 shows a non-limiting example of a fluorescence resonance energy transfer (FRET) solution phase assay format. In the absense of a sVEGFR target molecule, the nucleic acid sensor molecule is inactive. In the presense of a sVEGFR target molecule, the nucleic acid sensor molecule is active, and cleaves a substrate reporter molecule comprising a nucleic acid sequence having a fluorophore (D) and quencher moiety (A). Once the reporter molecule is cleaved, the distance between the fluorophore and quencher moiety is increased, resulting is fluorescence and signal generation. Different fluorophores (eg. Cy3, Rox, Cy5, and Cy7) have different wavelengths for detection, thereby allowing multiplexed assays for different targets within the same assay.

FIG. 4 shows a non-limiting example of a colorimetric solution phase assay format. In the absense of a sVEGFR target molecule, the nucleic acid sensor molecule is inactive. In the presense of a sVEGFR target molecule, the nucleic acid sensor molecule is active, and cleaves a substrate reporter molecule comprising a nucleic acid sequence having a terminal colorimetric group, such as a para-nitrophenyl group. Once the reporter molecule is cleaved, the colorimetric group is released (such as p-nitrophenol), generating a detectable color.

FIG. 5 shows a non-limiting diagrammatic example of a method for generating nucleic acid sensor molecules of the invention using a partially defined sequence comprising a known enzymatic nucleic acid molecule coupled with a randomized sensor region represented by Ns in the figure. The method comprises: (1) generating a pool of nucleic acid sequences comprising a fixed domain and a random domain, (2) discarding any active sequences that have catalytic activity in the absense of sVEGFR target, (3) adding sVEGFR target to the pool, (4) discarding molecules that are inactive in the presence of the sVEGFR target, (5) amplification to enrich nucleic acid sensor sequences, and (6) repeating the process of (1-5) to increase nucleic acid sensor sensitivity and catalytic activity.

FIG. 6 shows a non-limiting diagrammatic example of a method for generating nucleic acid sensor molecules of the invention using a defined aptamer sequence having specificity for the sVEGFR target molecule coupled to a known enzymatic nucleic acid molecule via a randomized stem sequence represented by Ns in the figure. The method comprises: (1) generating a pool of nucleic acid sequences comprising two fixed domains (aptamer sensor domain and enzymatic nucleic acid domain) and a random domain (connecting sequence), (2) discarding any active sequences that have catalytic activity in the absense of sVEGFR target, (3) adding sVEGFR target to the pool, (4) discarding molecules that are inactive in the presence of the sVEGFR target, (5) amplification to enrich nucleic acid sensor sequences, and (6) repeating the process of (1-5) to increase nucleic acid sensor sensitivity and catalytic activity.

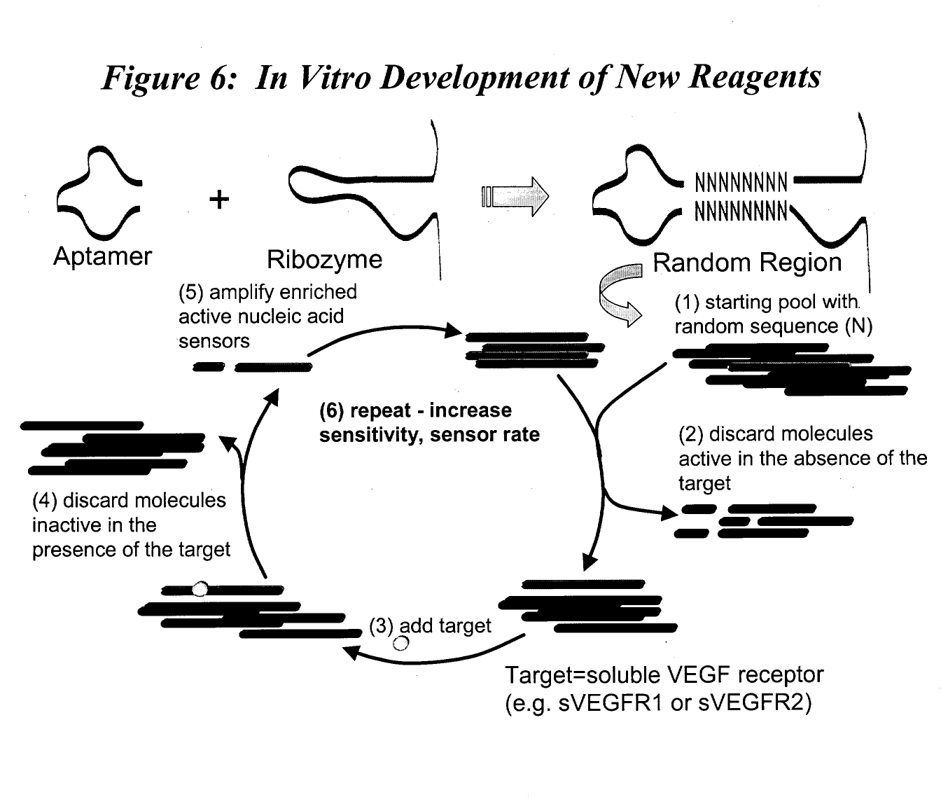

FIG. 7 shows a non-limiting example of an arrayed detection format using nucleic acid sensor molecules. In this example, the support bound nucleic acid sensor ligates a reporter signal molecule in the presence of a target molecule, thus generating a detectable signal. As such, detection assays can be multiplexed for various target molecules based on position or signal type readout.

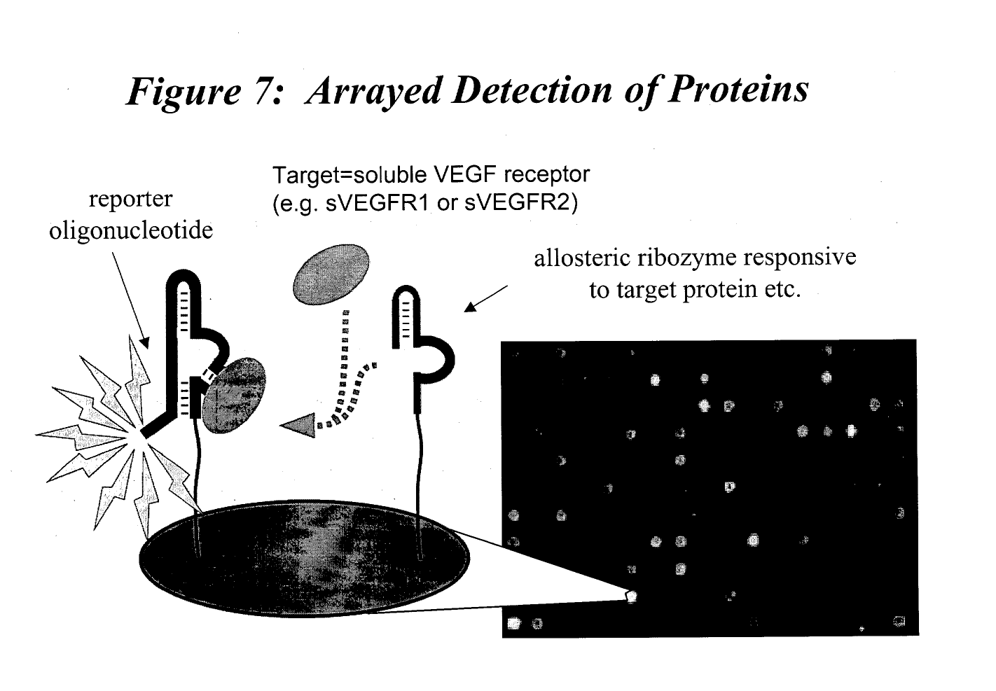

FIG. 8 shows the structure of ANGIOZYME, an enzymatic nucleic acid that targets VEGFR1, including sequence and modifications. ANGIOZYME is shown bound to its target site in VEGFR-1 RNA. An arrow indicates the cleavage site.

FIG. 9 shows a schematic representation of the treatment schedule of clinical protocol RPI0003.

FIG. 10 shows a graph describing the relationship of pretreatment dectecion of sVEGFR-1 and tumor response in patients enrolled in the RPI0003 study.

FIG. 11 shows a graph describing the relationship of pretreatment detection of sVEGFR-1 and time to progression in patients enrolled in the RPI0003 study.

FIG. 12 shows a graph describing the relationship of the disappearance of sVEGFR-1 by week 12 and tumor response in patients enrolled in the RPI0003 study.

FIG. 13 shows a graph describing the relationship of the disappearance of sVEGFR-1 and time to progression in patients enrolled in the RPI0003 study.

### DETAILED DESCRIPTION OF THE INVENTION

The present invention relates to generally to compounds and methods relating to detection of soluble VEGFR expression for generating prognostic criteria useful in establishing methods of treatment in cancer patients. In the method of the present invention, soluble VEGFR levels are utilized as a means by which cancer can be detected, efficacy of cancer therapies can be evaluated, prognosis of a subject can be predicted, new anti-cancer therapies can be discovered, treatment regimens can be determined, and personalized treatment regimens can be established. For example, subjects that are positive for sVEGFR expression can be identified for treatment with VEGF and/or VEGFR specific drugs. The invention features reagents and methods to differentiate cancerous conditions that are related to expression of soluble VEGFR and those that are not related to expression of soluble VEGFR. The invention therefore permits prediction of what therapeutic approaches can best be used to treat a subject having a phenotype characterized by soluble VEGFR expression. In addition, the invention provides a means to screen existing anti-cancer therapies and new anti-cancer therapies in drug discovery that can be used to treat a subject having a phenotype characterized by soluble VEGFR expression. Specifically, the present invention is based on the observation that the progression of a cancerous condition (e.g., colorectal cancer) can be distinguished using levels of soluble VEGFR observed in a subject.

Assays of the present invention provide rapid and inexpensive methods for the early identification of cancer in a subject. Importantly, it is contemplated that the methods of the present invention will permit rapid identification of VEGFR and/or VEGF phenotypes in uncharacterized subject populations. Moreover, the method of the present invention also provides a prognostic means to predict the progression of disease.

The present invention contemplates that the method of the present invention can be combined with other techniques to assess: (1) the level of membrane bound VEGFR by serum analysis of soluble VEGFR; (2), the predicted course of disease progression; (3) the efficacy of existing anti-cancer treatments; (4) the efficacy of new anti-cancer treatments; and (5) new strategies of personalized medicine.

The present invention permits study of the genes responsible for inducing angiogenesis in a subject. It is contemplated therefore, that the present invention will be useful for distinguishing various cancer types, including but not limited to, such cancers that are related to expression of VEGFR and/or VEGF (e.g., cancers having a soluble VEGFR phenotype). Specifically, the present invention provides a screening method useful to identify and test the effects of the genes which control and/or participate in angiogenesis useful in indentifying new therapeutic compounds that modulate expression of such genes. It is contemplated that these functions and/or genes will be targets for biological and/or pharmacological (e.g., pharmaceutical) intervention. Indeed, the present invention provides a method to screen for direct and/or side effects of biological and/or pharmacological compounds, in particular those which lead to decreased expression of VEGFR and/or VEGF by measuring the level of soluble VEGFR. It is contemplated that these compounds will be useful in treatment of angiogenesis related diseases and disorders. It is also contemplated that the present invention will be useful in studies to reverse and/or delay angiogenesis in cancerous cells and/or tissues.

The present invention also contemplates a rapid, convenient, inexpensive diagnostic and/or prognostic assay for evaluation of diseases associated with high VEGFR and/or VEGF expression by measuring the level of soluble VEGFR. It also contemplates an assay for assessment of the extent to which a subject will respond to a given treatment or course of therapy. The present invention also contemplates a rapid, convenient, inexpensive assay for the in vivo assessment in humans, other animals, and/or animal models for the anti-angiogenic compounds or treatment regimens.

The term “soluble VEGFR”, or “sVEGFR” as used herein refers to the variant form of VEGFR that can be generated by differential splicing of the VEGFR mRNA that results in the soluble form of VEGFR as opposed to membrane bound VEGFR. As used herein, soluble VEGFR is not limited to the full length VEGFR protein, but also includes any subunits or polypeptide and peptide fragments thereof.

The term “VEGFR” as used herein refers to a vascular endothelial growth factor receptor, for example VEGFR1, VEGFR2, and/or VEGFR3. In preferred embodiments of the invention, the methods and compositions of the invention relate to detection and/or analysis of VEGFR1 levels.

The term “detecting” is used in the broadest sense to include both qualitative and quantitative measurements of a target molecule (e.g. soluble VEGFR such as sVEGFR1). In one embodiment, the method is used to determine whether soluble VEGFR in a sample is at a detectable level. In another embodiment, the method can be used to quantify the amount of soluble VEGFR in a sample and further to compare the soluble VEGFR levels from different samples.

The term “sample” or “biological sample” refers generally to a body sample from any animal such as a mammal or human. In one embodiment, the sample is from cancer, vascular, or diabetic subjects. Such samples include biological fluids such as serum, plasma, vitreous fluid, lymph fluid, synovial fluid, follicular fluid, seminal fluid, amniotic fluid, milk, whole blood, urine, cerbro-spinal fluid, saliva, sputum, tears, perspiration, mucus, and tissue culture medium, as well as tissue extracts such as homogenized tissue, and cellular extracts.

The term “capture reagent” refers to a reagent capable of binding and capturing a target molecule (e.g. soluble VEGFR) in a sample such that under suitable conditions, the capture reagent-target molecule complex can be separated from the rest of the sample or detected directly in the sample. Typically, the capture reagent is immobilized or immobilizable. For example, in a sandwich immunoassay, the capture reagent is preferably an antibody or a mixture of different antibodies against a target antigen.

The term “detectable antibody” refers to an antibody that is capable of being detected either directly through a label amplified by detection means, or indirectly through, for example, another antibody that is labeled. For direct labeling, the antibody is typically conjugated to a moiety that is detectable by some means, such as a biotinylated antibody.

The term “antibody” is used in the broadest sense and includes monoclonal antibodies (including agonist, antagonist, and neutralizing antibodies), polyclonal antibodies, multivalent antibodies, multispecific antibodies, and antibody fragments so long as they exhibit the desired binding capacity.

The term “monoclonal antibody” as used herein refers to an antibody obtained from a population of substantially homogeneous antibodies, i.e., the individual antibodies comprising the population are identical except for possible naturally-occurring mutations that can be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen.

The monoclonal antibodies herein include hybrid and recombinant antibodies produced by splicing a variable (including hypervariable) domain of an anti-VRP antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments so long as they exhibit the desired biological activity.

Thus, the modifier “monoclonal” indicates the character of the antibody as being obtained from a substantially homogeneous population of antibodies, and is not to be construed as requiring production of the antibody by any particular method.

“Humanized” forms of non-human (e.g. murine) antibodies are specific chimeric immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin.

“Treatment” refers to both therapeutic treatment and prophylactic or preventative measures using various therapies. Those in need of treatment include those already with the disorder as well as those prone to have the disorder or those in which the disorder is to be prevented.

By “therapy” or “therapeutic” is meant a method or composition that is used to treat a disease or condition. Such therapies include but are not limited to treatment with small molecules (e.g. chemotherapy), antibodies, nucleic acid molecules (e.g. siNA, aptamers, enzymatic nucleic acids), proteins, polypeptides, peptides, hormones, and/or other biologically active molecules in addition to radiation treatments, surgery, nutritional modification, and/or behavioral modification. Such therapies can be monotherapies or combination therapies in which two or more therapies are combined for improved efficacy or response.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). Non limiting examples of siNA molecules of the invention are described in Haeberli et al., PCT/US03/05346 and McSwiggen et al., PCT/US03/05028, both incorporated by reference herein in their entirety including the drawings. Chemical modifications described in Haeberli et al., PCT/US03/05346 and McSwiggen et al., PCT/US03/05028 can be applied to any siNA sequence of the invention. For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiment, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), translational silencing, and others. In addition, as used herein, the term RNAi is meant to be equivalent to other terms used to describe sequence specific RNA interference, such as post transcriptional gene silencing, or epigenetics. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure to alter gene expression (see, for example, Allshire, 2002, Science, 297, 1818-1819; Volpe et al., 2002, Science, 297, 1833-1837; Jenuwein, 2002, Science, 297, 2215-2218; and Hall et al.; 2002, Science, 297, 2232-2237).

By “enzymatic nucleic acid” is meant a nucleic acid molecule capable of catalyzing (altering the velocity and/or rate of) a variety of reactions including the ability to repeatedly cleave other separate nucleic acid molecules (endonuclease activity) or ligate other separate nucleic acid molecules (ligation activity) in a nucleotide base sequence-specific manner. Additional reactions amenable to enzymatic nucleic acid molecules include but are not limited to phosphorylation, dephosphorylation, isomerization, helicase activity, polymerization, transesterification, hydration, hydrolysis, alkylation, dealkylation, halogenation, dehalogenation, esterification, desterification, hydrogenation, dehydrogenation, saponification, desaponification, amination, deamination, acylation, deacylation, glycosylation, deglycosylation, silation, desilation, hydroboration, epoxidation, peroxidation, carboxylation, decarboxylation, substitution, elimination, oxidation, and reduction reactions on both small molecules and macromolecules.

Such a molecule with endonuclease and/or ligation activity can have complementarity in a substrate binding region to a specified gene target, and also has an enzymatic activity that specifically cleaves and/or ligates RNA or DNA in that target. That is, the nucleic acid molecule with endonuclease and/or ligation activity is able to intramolecularly or intermolecularly cleave and/or ligate RNA or DNA and thereby inactivate or activate a target RNA or DNA molecule. This complementarity functions to allow sufficient hybridization of the enzymatic RNA molecule to the target RNA or DNA reporter molecule to allow the cleavage/ligation to occur. 100% complementarity is preferred, but complementarity as low as 50-75% can also be useful in this invention.

In addition, a nucleic acid sensor molecule, such as an allozyme, can perform other reactions, including those mentioned above, selectively on both small molecule and macromolecular substrates, though specific interaction of the nucleic acid sensor molecule sequence with the desired substrate molecule via hydrogen bonding, electrostatic interactions, and Van der Waals interactions.

The nucleic acids of the compounds of the invention can be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme, finderon or DNA enzyme. All of these terminologies describe nucleic acid molecules with enzymatic activity.

There are several different structural motifs of enzymatic nucleic acid molecules that catalyze cleavage/ligations reaction, including but not limited to hammerhead motif, hairpin motif, hepatitis delta virus motif, G-cleaver motif, Amberzyme motif, inozyme motif, and Zinzyme motif. Other motifs can be evolved using in vitro or in vivo selection techniques.

By “substrate binding arm” or “substrate binding domain” or “substrate binding region” is meant that portion or region of a nucleic acid sensor molecule which is able to interact, for example, via complementarity (i.e., able to base-pair with), with a portion of its substrate or reporter. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). That is, these arms contain sequences within a nucleic acid sensor molecule which are intended to bring the nucleic acid sensor molecule and the reporter molecule, for example RNA, together through complementary base-pairing interactions. The nucleic acid sensor molecule of the invention can have binding arms that are contiguous or non-contiguous and can be of varying lengths. The length of the binding arm(s) are preferably greater than or equal to four nucleotides and of sufficient length to stably interact with the target reporter sequence. Preferably, the binding arm(s) are 12-100 nucleotides in length. More preferably, the binding arms are 14-24 nucleotides in length (see, for example, Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “enzymatic portion” or “catalytic domain” is meant that portion or region of the nucleic acid sensor molecule essential for catalyzing a chemical reaction, such as cleavage of a nucleic acid substrate.

By “system” or “sample” is meant material, in a purified or unpurified form, from biological or non-biological sources, including but not limited to human, animal, soil, food, water, or others sources that comprise the target signaling agent or target signaling molecule to be detected. As such, nucleic acid sensor molecules and aptamers of the invention can be used to assay target compounds in biologic and non-biologic systems, such as in human and animal subjects or in samples of unidentified materials outside of a biological system.

The “biological system” or “biological sample” as used herein can be a eukaryotic system or a prokaryotic system, for example a bacterial cell, plant cell or a mammalian cell, or of plant origin, mammalian origin, yeast origin, Drosophila origin, or archebacterial origin.

By “reporter molecule” is meant a molecule, such as a nucleic acid sequence (e.g., RNA or DNA or analogs thereof) or peptides and/or other chemical moieties, able to stably interact with the nucleic acid sensor molecule and function as a substrate for the nucleic acid sensor molecule. The reporter molecule can be covalently linked to the nucleic acid sensor molecule or a portion of one of the components of a halfzyme. The reporter molecule can also contain chemical moieties capable of generating a detectable response, including but not limited to, fluorescent, chromogenic, radioactive, enzymatic and/or chemiluminescent or other detectable labels that can then be detected using standard assays known in the art. The reporter molecule can also act as an intermediate in a chain of events, for example, by acting as an amplicon, inducer, promoter, or inhibitor of other events that can act as second messengers in a system.

In one embodiment, the reporter molecule of the invention is an oligonucleotide primer, template, or probe, which can be used to modulate the amplification of additional nucleic acid sequences, for example, sequences comprising reporter molecules, target signaling molecules, effector molecules, inhibitor molecules, and/or additional nucleic acid sensor molecules of the instant invention.

By “sensor component” or “sensor domain” of the nucleic acid sensor molecule is meant, a molecule such as a nucleic acid sequence (e.g., RNA or DNA or analogs thereof), peptide, or other chemical moiety which can interact with one or more regions of a target signaling agent or more than one target signaling agents, and which interaction causes the enzymatic nucleic acid component of the nucleic acid sensor molecule to modulate, such as inhibit or activate, the catalytic activity of the nucleic acid sensor molecule. In the presence of a signaling agent, the ability of the sensor component, for example, to modulate the catalytic activity of the enzymatic nucleic acid component is inhibited or diminished. The sensor component can comprise recognition properties relating to chemical or physical signals capable of modulating the enzymatic nucleic acid component via chemical or physical changes to the structure of the nucleic acid sensor molecule. The sensor component can also be derived from a nucleic acid sequence that is obtained through in vitro or in vivo selection techniques as are know in the art.

Alternately, the sensor component can be derived from a nucleic acid molecule (aptamer) which is evolved to bind to a nucleic acid sequence within a target nucleic acid molecule. Such sequences or aptamers can be designed to bind a specific protein (e.g. VEGFR), peptide, nucleic acid, co-factor, metabolite, and/or associated antibody or other molecule with varying affinity. The sensor component can be covalently linked to the nucleic acid sensor molecule, or can be non-covalently associated. A person skilled in the art will recognize that all that is required is that the sensor component is able to selectively inhibit the activity of the nucleic acid sensor molecule.

By “aptamer” or “nucleic acid aptamer” as used herein is meant a nucleic acid molecule that binds specifically to a target molecule wherein the nucleic acid molecule has sequence that is distinct from sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628.

“Complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, ligation, isomerization, phosphorylation, or dephosphorylation. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “alkyl” group is meant a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) are preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) can be preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH. Such alkyl groups can also include aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” is meant a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “nucleoside” is meant a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an nucleic acid sensor molecule and/or in the substrate-binding regions of the nucleic acid molecule.

By “unmodified nucleotide” is meant a nucleotide with one of the bases adenine, cytosine, guanine, thymine, uracil joined to the 1′ carbon of beta-D-ribo-furanose.

By “modified nucleotide” is meant a nucleotide that contains a modification in the chemical structure of an unmodified nucleotide base, sugar and/or phosphate.

By “unmodified nucleoside” is meant a nucleoside with one of the bases adenine, cytosine, guanine, thymine, uracil joined to the 1′ carbon of beta-D-ribo-furanose.

By “modified nucleoside” is meant a nucleotide that contains a modification in the chemical structure of an unmodified nucleoside base or sugar.

By “sufficient length” is meant an oligonucleotide of length sufficient to provide the intended function (such as binding) under the expected condition. For example, a binding arm of the enzymatic nucleic acid component of the nucleic acid sensor molecule should be of “sufficient length” to provide stable binding to the reporter molecule under the expected reaction conditions and environment to catalyze a reaction. In a further example, the sensor domain of the nucleic acid sensor molecule should be of sufficient length to interact with a target nucleic acid molecule in a manner that would cause the nucleic acid sensor to be active.

By “stably interact” is meant interaction of the oligonucleotides with target nucleic acid (e.g., by forming hydrogen bonds with complementary nucleotides in the target under physiological conditions) that is sufficient for the intended purpose (e.g., cleavage of target RNA by an enzyme).

By “nucleic acid molecule” as used herein is meant a molecule comprising nucleotides. The nucleic acid can be single, double, or multiple stranded and can comprise modified or unmodified nucleotides or non-nucleotides or various mixtures and combinations thereof. Nucleic acid molecules shall include oligonucleotides, ribozymes, DNAzymes, templates, and primers.

By “oligonucleotide” is meant a nucleic acid molecule comprising a stretch of three or more nucleotides.

In one embodiment, the linker region, when present in the nucleic acid sensor molecule and/or reporter molecule is further comprised of nucleotide, non-nucleotide chemical moieties or combinations thereof. Non-limiting examples of non-nucleotide chemical moieties can include ester, anhydride, amide, nitrile, and/or phosphate groups.

In another embodiment, the non-nucleotide linker is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. Thus, in one embodiment, the invention features a nucleic acid sensor molecule of the invention having one or more non-nucleotide moieties, and having enzymatic activity to perform a chemical reaction, for example to cleave an RNA or DNA molecule.

By “cap structure” is meant chemical modifications which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both termini. In non-limiting examples: the 5′-cap is selected from the group comprising inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein). In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

The term “non-nucleotide” refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenine, guanine, cytosine, uracil or thymine.

The terms “abasic” or “abasic nucleotide” are meant to include sugar moieties lacking a base or having other chemical groups in place of a base at the 1′ position, (for more details see Wincott et al., International PCT publication No. WO 97/26270).

By “RNA” is meant a molecule comprising at least one ribonucleotide residue. By “ribonucleotide” or “2′-OH” is meant a nucleotide with a hydroxyl group at the 2′ position of a β-D-ribo-furanose moiety.

By “subject” is meant an organism, which is a donor or recipient of explanted cells or the cells themselves. “Subject” also refers to an organism to which the nucleic acid molecules of the invention can be administered. A subject can be a mammal or mammalian cells. A subject cab be a human or human cells. The term “subject” can be used interchangeably with the term “patient” herein.

By “enhanced enzymatic activity” is meant to include activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the individual catalytic activity or stability of the nucleic acid molecule can be decreased (i.e., less than ten-fold), but the overall activity of the nucleic acid molecule is enhanced, in vivo.

By “detectable response” is meant a chemical or physical property that can be measured, including, but not limited to changes in temperature, pH, frequency, charge, capacitance, or changes in fluorescent, chromogenic, colorimetric, radioactive, enzymatic and/or chemiluminescent levels or other properties that can then be detected using standard methods discussed herein and known in the art.

By “predetermined target” is meant a signaling agent or target signaling agent that is chosen to interact with a nucleic acid sensor molecule to generate a detectable response, such as sVEGFR.

Nucleic Acid Molecule Synthesis

The nucleic acid molecules of the invention, including certain nucleic acid sensor molecules, can be synthesized using the methods described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59. Such methods make use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table I outlines the amounts and the contact times of the reagents used in the synthesis cycle. Alternatively, syntheses at the 0.2 μmol scale can be done on a 96-well plate synthesizer, such as the PG2100 instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Cleavage from the solid support and deprotection of the oligonucleotide is typically performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA.3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃. An alternative deprotection cocktail for use in the one pot protocol comprises the use of aqueous methylamine (0.5 ml) at 65° C. for 15 min followed by DMSO (0.8 ml) and TEA.3HF (0.3 ml) at 65° C. for 15 min. A similar methodology can be employed with 96-well plate synthesis formats by using a Robbins Scientific Flex Chem block, in which the reagents are added for cleavage and deprotection of the oligonucleotide.

For anion exchange desalting of the deprotected oligomer, the TEAB solution is loaded onto a Qiagen 500® anion exchange cartridge (Qiagen Inc.) that is prewashed with 50 mM TEAB (10 mL). After washing the loaded cartridge with 50 mM TEAB (10 mL), the RNA is eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile. Alternatively, for oligonucleotides synthesized in a 96-well format, the crude trityl-on oligonucleotide is purified using a 96-well solid phase extraction block packed with C18 material, on a Bahdan Automation workstation.

The average stepwise coupling yields are typically >98% (Wincott et al., 1995 Nucleic Acids Res. 23, 2677-2684). Those of ordinary skill in the art will recognize that the scale of synthesis can be adapted as larger or smaller than the example described above including but not limited to 96 well format, all that is important is the ratio of chemicals used in the reaction.

To ensure the quality of synthesis of nucleic acid molecules of the invention, quality control measures are utilized for the analysis of nucleic acid material. Capillary Gel Electrophoresis, for example using a Beckman MDQ CGE instrument, can be ulitized for rapid analysis of nucleic acid molecules, by introducing sample on the short end of the capillary. In addition, mass spectrometry, for example using a PE Biosystems Voyager-DE MALDI instrument, in combination with the Bohdan workstation, can be utilized in the analysis of oligonucleotides, including oligonucleotides synthesized in the 96-well format.

The nucleic acids of the invention can also be synthesized in two parts and annealed to reconstruct the nucleic acid sensor molecules (Chowrira and Burke, 1992 Nucleic Acids Res., 20, 2835-2840). The nucleic acids are also synthesized enzymatically using a variety of methods known in the art, for example as described in Havlina, International PCT publication No. WO 9967413, or from DNA templates using bacteriophage T7 RNA polymerase (Milligan and Uhlenbeck, 1989, Methods Enzymol. 180, 51). Other methods of enzymatic synthesis of the nucleic acid molecules of the invention are generally described in Kim et al., 1995, Biotechniques, 18, 992; Hoffman et al., 1994, Biotechniques, 17, 372; Cazenare et al., 1994, PNAS USA, 91, 6972; Hyman, U.S. Pat. No. 5,436,143; and Karpeisky et al., International PCT publication No. WO 98/28317).

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention can be modified to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Nucleic acid sensor molecules are purified by gel electrophoresis using known methods or are purified by high pressure liquid chromatography (HPLC; See Wincott et al., Supra, the totality of which is hereby incorporated herein by reference) and are re-suspended in water.

Optimizing Nucleic Acid Molecule Activity

Synthesizing nucleic acid molecules with modifications (base, sugar and/or phosphate) that prevent their degradation by serum ribonucleases can increase their potency (see e.g., Eckstein et al., International Publication No. WO92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO93/15187; Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules described herein. All these references are incorporated by reference herein. Modifications which enhance their efficacy in cells, and removal of bases from nucleic acid molecules to shorten oligonucleotide synthesis times and reduce chemical requirements are preferably desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modifications of nucleic acid molecules have been extensively described in the art (see Eckstein et al., International Publication PCT No. WO 92/07065; Perrault et al. Nature, 1990, 344, 565-568; Picken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated by reference herein in their totalities). Such publications describe general methods and strategies to determine the location of incorporation of sugar, base and/or phosphate modifications and the like into nucleic acid sensor molecule molecules without inhibiting catalysis. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications which maintain or enhance activity are provided. Such nucleic acid is also generally more resistant to nucleases than unmodified nucleic acid. Thus, in the presence of biological fluids, or in cells, the activity can not be significantly lowered. Clearly, nucleic acid molecules must be resistant to nucleases in order to function as effective diagnostic agents, whether utilized in vitro and/or in vivo. Improvements in the synthesis of RNA and DNA (Wincott et al., 1995 Nucleic Acids Res. 23, 2677; Caruthers et al., 1992, Methods in Enzymology 211,3-19; Karpeisky et al., International PCT publication No. WO 98/28317) (incorporated by reference herein) have expanded the ability to modify nucleic acid molecules by introducing nucleotide modifications to enhance their nuclease stability as described above.

In another aspect the nucleic acid molecules comprise a 5′ and/or a 3′-cap structure.

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Karpeisky et al., WO 98/28317, respectively, which are both incorporated by reference herein in their entireties.

Various modifications to nucleic acid (e.g., nucleic acid sensor molecule) structure can be made to enhance the utility of these molecules. Such modifications enhance shelf-life, half-life in vitro, stability, and ease of introduction of such oligonucleotides to the target site, e.g., to enhance penetration of cellular membranes, and confer the ability to recognize and bind to targeted cells.

### EXAMPLES

The following examples describe non-limiting examples of methods of the invention useful in providing reagents and assays of the invention, useful in practicing methods of the instant invention.

### Example 1

sVEGFR Aptamer Selection

A nucleic acid aptamer that selectively binds human soluble VEGFR (sVEGFR) is provided in accordance with the present invention. The binding affinity of the aptamer for these compounds is preferably represented by the dissociation constant of about 50 nanomolar (nM) or less, and more preferably about 10 nM or less. In one embodiment, the Kd of the aptamer and target is established using a double filter nitrocellulose filter binding assay such as that disclosed by Wong and Lohman, 1993, PNAS USA, 90, 5428-5432.

Generally, the method for isolating aptamers of the invention having specificity for sVEGFR comprises: (a) preparing a candidate mixture of potential oligonucleotide ligands for sVEGFR wherein the candidate mixture is complex enough to contain at least one oligonucleotide ligand for sVEGFR (the target); (b) contacting the candidate mixture with the target under conditions suitable for at least one oligonucleotide in the candidate mixture to bind to the target; (c) removing unbound oligonucleotides from the candidate mixture; (d) collecting the oligonucleotide ligands that are bound to the target to produce a first collected mixture of oligonucleotide ligands; (e) contacting the mixture from (d) with the target under more stringent binding conditions than in (b), wherein oligonucleotide ligands having increased affinity to the target relative to the first collected mixture of (d); (f) removing unbound oligonucleotides from (e); and (g) collecting the oligonucleotide ligands that are bound to the target to produce a second collected mixture of oligonucleotide ligands to thereby identify oligonucleotides having specificity for sVEGFR. The method can comprise additional steps in which the oligonucleotides isolated in the first or second collected mixture are enriched or expanded by any suitable technique, such as amplification or mutagenesis, prior to contacting the first collected oligonucleotide mixture with the target under the higher stringency conditions, after collecting the oligonucleotides that bound to the target under the higher stringency conditions, or both. Optionally, the contacting and expanding or enriching steps are repeated as necessary to produce the desired aptamer. Thus, it is possible that the second collected oligonucleotide mixture can comprise a single aptamer. The conditions used to affect the stringency of binding used in the method can include varying reaction conditions used for binding, for example the composition of a buffer, temperature, time, and concentration of the components used for binding can be optimized for the desired level of stringency.

In vitro Selection

In a non-limiting example, aptamers having binding specificity for a sVEGFR target are isolated by applying the method under the following conditions. First, the sVEGFR target is attached to a solid matrix such as a bead or chip surface by means of a covalent (eg. amide or morpholino bond) or non-covalent (eg. biotin/streptavidin) linkage.

A random pool of DNA oligomers is synthesized where the 5′ and 3′ proximal ends are fixed sequences used for amplification and the central region consists of randomized positions. Ten picomoles of template are PCR amplified for 8 cycles in the initial round. Copy DNA of the selected pool of RNA from subsequent rounds of amplification are PCR amplified 18 cycles. PCR reactions are carried out in a 50 μl volume containing 200 picomoles of each primer, 2 mM final concentration dNTP's, 5 units of Thermus aquaticus DNA polymerase (Perkin Elmer Cetus) in a PCR buffer (10 mM Tris-Cl pH 8.4, 50 mM KCl, 7.5 mM MgCl₂, 0.05 mg/ml BSA). Primers are annealed at 58° C. for 20 seconds and extended at 74° C. for 2 minutes. Denaturation can occur at 93° C. for 30 seconds.

Products from PCR amplification are used for T7 in vitro transcription in a 200 ul reaction volume. T7 transcripts are purified from an 8 percent, 7M Urea polyacrylamide gel and eluted by crushing gel pieces in a Sodium Acetate/EDTA solution. For each round of amplification, 50 picomoles of the selected pool of RNA is phosphatased for 30 minutes using Calf Intestinal Alkaline Phosphatase. The reaction is then phenol extracted 3 times and chloroform extracted once, then ethanol precipitated. 25 picomoles of this RNA is 5′ end-labeled using γ-³²P ATP with T4 polynucleotide kinase for 30 minutes. Kinased RNA is gel purified and a small quantity (about 150 fmoles; 100,000 cpm) is used along with 250 picomoles of cold RNA to follow the fraction of RNA bound to the ecstasy target and retained on nitrocellulose filters during the separation step of the method. Typically a target molecule concentration is used that binds one to five percent of the total input RNA. A control (without target) is used to determine the background which is typically 0.1% of the total input. Selected RNA is eluted from the filter by extracting three times with water saturated phenol containing 2% lauryl sulfate (SDS), 0.3M NaOAc and 5 mM EDTA followed by a chloroform extraction. Twenty five percent of this RNA is then used to synthesize cDNA for PCR amplification.

Selection with Non-Amplifiable Competitor RNA

In a non-limiting example, selections are performed using two buffer conditions where the only difference between the buffers is sodium concentration (250 mM NaCl or 500 mM NaCl). Two different buffer conditions are used to increase stringency (with the higher salt concentration being more stringent) and to determine whether different ligands can be obtained. After 10 rounds of amplification, the binding constant of the selected pool can decrease by about an order of magnitude and can remain constant for the next two additional rounds. Competitor RNA is not used in the first 12 rounds. After this round, the pool is split and selection carried out in the presence and absence (control) of competitor RNA. For rounds 12 through 18, a 50-fold excess of a non-amplifiable random pool of RNA is present during selection to compete with non-specific low-affinity binders that may survive and thus be amplified. The competitor RNA, which had a 30N random region, is made as described above for the amplifiable pool RNA; however, the competitor RNA has different primer annealing sequences. Thus, the competitor RNA does not survive the cDNA synthesis or PCR amplification steps. It would be apparent to one skilled in the art that other primer sequences could be used as long as they are not homologous to those used for the pool RNA. The use of competitor RNA can increase the affinity of the selected pool by several orders of magnitude.

Cloning and Sequencing

In a non-limiting example, PCR amplified DNA from the last round selected-pool of RNA is phenol and chloroform extracted and ethanol precipitated. The extracted PCR DNA is then digested using Bam HI and Hind III restriction enzymes and sub-cloned into pUC18. DNAs are phenol and chloroform extracted following digestion. Ligation is carried out at room temperature for two hours after which time the reaction is phenol and chloroform extracted and used to electroporate competent cells. Fifty transformants from the selections using competitor RNA at both NaCl concentrations are picked and their DNA is sequenced.

Binding Assays

In a non-limiting example, binding assays were performed by adding 5 ul of the target, at the appropriate concentrations (i.e., ranging from 2×10⁻⁶ with 3 fold dilutions to 9×10⁻⁹ for 250 mM NaCl and 0.5×10^{31 7} with 3 fold dilutions to 2×10⁻¹⁰ for 50 mM NaCl), to 45 ul of binding buffer (50 mM Na-HEPES pH 7.5, 250 mM NaCl, 2 mM DTT, 10 mM MnCl₂, 5 mM CHAPS) on ice, then adding 50,000 cpm of kinased RNA (<200 fmoles) in a volume of 3 to 4 ul. This mix was incubated at 37° C. for 20 minutes. The reactions were then passed over nitrocellulose filters, which are pre-equilibrated in buffer, and washed with a 50 mM Tris-Cl pH 7.5 solution. Filters were dried and counted.

General Considerations in Aptamer Selection

When a consensus sequence is identified, oligonucleotides that contain that sequence can be made by conventional synthetic or recombinant techniques. These aptamers can also function as target-specific aptamers of this invention. Such an aptamer can conserve the entire nucleotide sequence of an isolated aptamer, or can contain one or more additions, deletions or substitutions in the nucleotide sequence, as long as a consensus sequence is conserved. A mixture of such aptamers can also function as target-specific aptamers, wherein the mixture is a set of aptamers with a portion or portions of their nucleotide sequence being random or varying, and a conserved region that contains the consensus sequence. Additionally, secondary aptamers can be synthesized using one or more of the modified bases, sugars and linkages described herein using conventional techniques and those described herein.

In some embodiments of this invention, aptamers can be sequenced or mutagenized to identify consensus regions or domains that are participating in aptamer binding to target, and/or aptamer structure. This information is used for generating second and subsequent pools of aptamers of partially known or predetermined sequence. Sequencing used alone or in combination with the retention and selection processes of this invention, can be used to generate less diverse oligonucleotide pools from which aptamers can be made. Further selection according to these methods can be carried out to generate aptamers having preferred characteristics for diagnostic or therapeutic applications. That is, domains that facilitate, for example, drug delivery could be engineered into the aptamers selected according to this invention.

Although this invention is directed to making aptamers using screening from pools of non-predetermined sequences of oligonucleotides, it also can be used to make second-generation aptamers from pools of known or partially known sequences of oligonucleotides. A pool is considered diverse even if one or both ends of the oligonucleotides comprising it are not identical from one oligonucleotide pool member to another, or if one or both ends of the oligonucleotides comprising the pool are identical with non-identical intermediate regions from one pool member to another. Toward this objective, knowledge of the structure and organization of the target protein can be useful to distinguish features that are important for biochemical pathway inhibition or biological response generation in the first generation aptamers. Structural features, such as secondary and tertiary structure, including, stems, loops, grooves, and the like can be considered in generating a second (less random) pool of oligonucleotides for generating second round aptamers.

### Example 2

Nucleic Acid Sensor Design Selection

The isolated apatmer obtained from in vitro selection is coupled to the stem-loop II region of a hammerhead ribozyme or to a region of another enzymatic nucleic acid motif using a randomized region of nucleotides. An enzymatic nucleic acid molecule where one or more regions are randomized is used. In this non-limiting example, in vitro selection is applied using a partially randomized RNA population based on the hammerhead self-cleaving ribozyme. The RNA construct used to express the population is designed to take advantage of the fact that the hammerhead ribozyme activity is sensitive to the structure of stem II. In this construct, stem II is replaced with two random-sequence domains that are separated by the aptamer sequence isolated above (sensor domain).

### Example 3

Selection Protocol for Isolation of sVEGFR Nucleic Acid Sensor Molecules

Negative Selection: The starting RNA population is comprised of greater than 1012 sequence variants. The RNA population is combined (final concentration=10 μM) with reaction buffer (50 mM Tris-HCl [pH 7.5 at 23° C.]; 20 mM MgCl₂) and incubated at 23° C. for 15 hr and the reaction products separated by denaturing (7 M urea) 10% polyacrylamide gel electrophoresis (PAGE). The uncleaved RNA is isolated by excising the precursor (uncleaved RNA) band and the RNA is recovered by a standard crush/soak method. The resulting RNA is precipitated using ethanol and the dried pellet resuspended in deionized water (dH2O).

Positive Selection: The negative-selected RNA population is combined (final concentration=10 μM) with reaction buffer (50 mM Tris-HCl [pH 7.5 at 23° C.]; 20 mM MgCl2). The VEGFR effector molecule is then added (final concentration of 1 μM) to initiate the reaction comprising incubation at 23° C. for 15 min and the reaction products separated by denaturing PAGE. The cleaved RNA is isolated by excising the appropriate cleavage product band and recovering the RNA by a standard crush/soak method. The resulting RNA is precipitated using ethanol and the dried pellet resuspended in deionized water (dH2O).

Amplification: Reverse transcription and polymerase chain reaction (RT-PCR) protocols are conducted according to standard methods. The resulting double-stranded DNA is used as template for in vitro transcription with T7 RNA polymerase under standard reaction conditions.

Protocol Variations. Various parameters of the protocol can be altered to apply selective pressure on specific characteristics of the nucleic acid sensor molecules. For example, decreasing incubation time during positive selection favors the isolation of nucleic acid sensor molecules with higher rate constants when bound to the effector. Increasing incubation time during negative selection favors the isolation of nucleic acid sensor molecules that have lower rate constants for nucleic acid sensor molecule cleavage in the absence of effector. Lowering the effector concentration favors the isolation of nucleic acid sensor molecules with improved affinity for the effector.

In the current example, early rounds of selection use 15 minute incubation for the positive selection reaction. This is progressively reduced to favor the isolation of higher-speed nucleic acid sensor molecules. Also, early rounds of selection make use of a separate reaction buffer wherein Tris is added first, effector is added next, and Mg2+ is added last. This protocol gives rise to a population of nucleic acid sensor molecules that largely requires this order of addition (nucleic acid sensor molecules do not become active if Tris and Mg2+ are added in combination, followed by addition of effector). In later rounds, the order of addition is altered as outlined above, and this change permitted the selection of nucleic acid sensor molecules that are able to switch from the OFF state to the ON state when effector is applied.

An essential component of the selection process is the use of modified negative selection protocols that disfavor the isolation of selfish molecules. For example, in later rounds, negative selection reactions are employed that comprise repetitive cycles (3 to 5) of ˜1 hr incubation at 23° C. followed by a 30 sec incubation at 90° C. This is to permit misfolded RNAs to become denatured and refolded in order to maximize the removal of nucleic acid sensor molecules that do not require effector to cleave.

### Example 4

Selection of sVEGFR Dependent Nucleic Acid Sensor Molecules

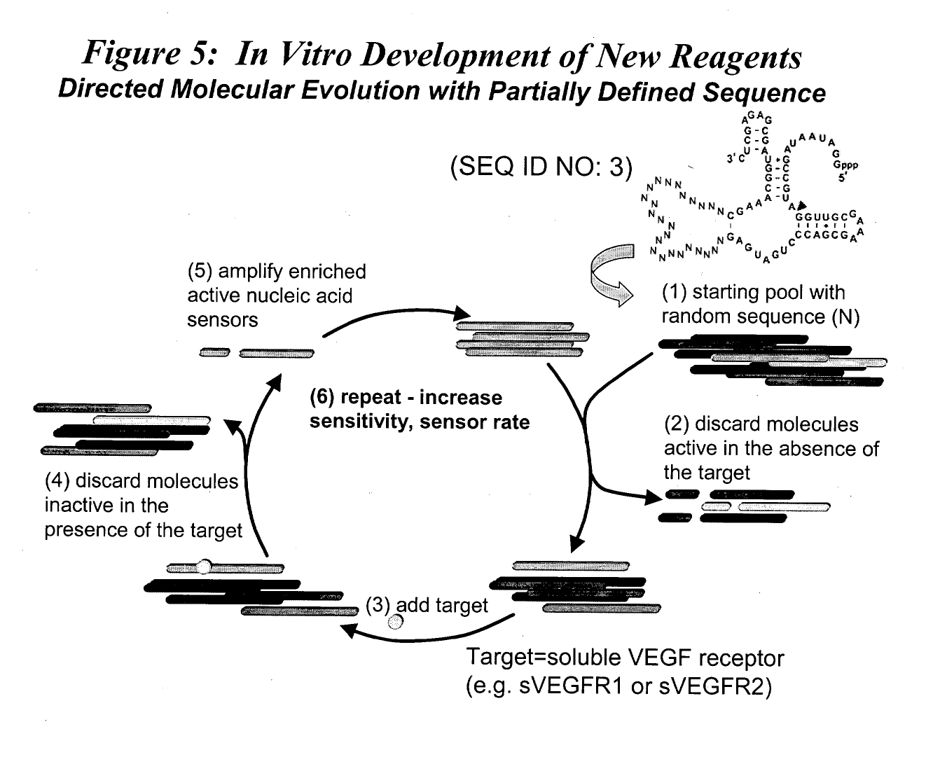

A nucleic acid sensor molecule is generated by in vitro selection techniques to be active only in the presence of the target molecule, such as sVEGFR. In this non-limiting example, in vitro selection is applied using a partially randomized RNA population based on the hammerhead self-cleaving ribozyme (FIG. 5). The RNA construct used to express the population is designed to take advantage of the fact that the hammerhead ribozyme activity is sensitive to the structure of stem II. In this construct, stem II is replaced with one or more randomized sensor domain.

### Example 5

Detection of sVEGFR in Biological Samples

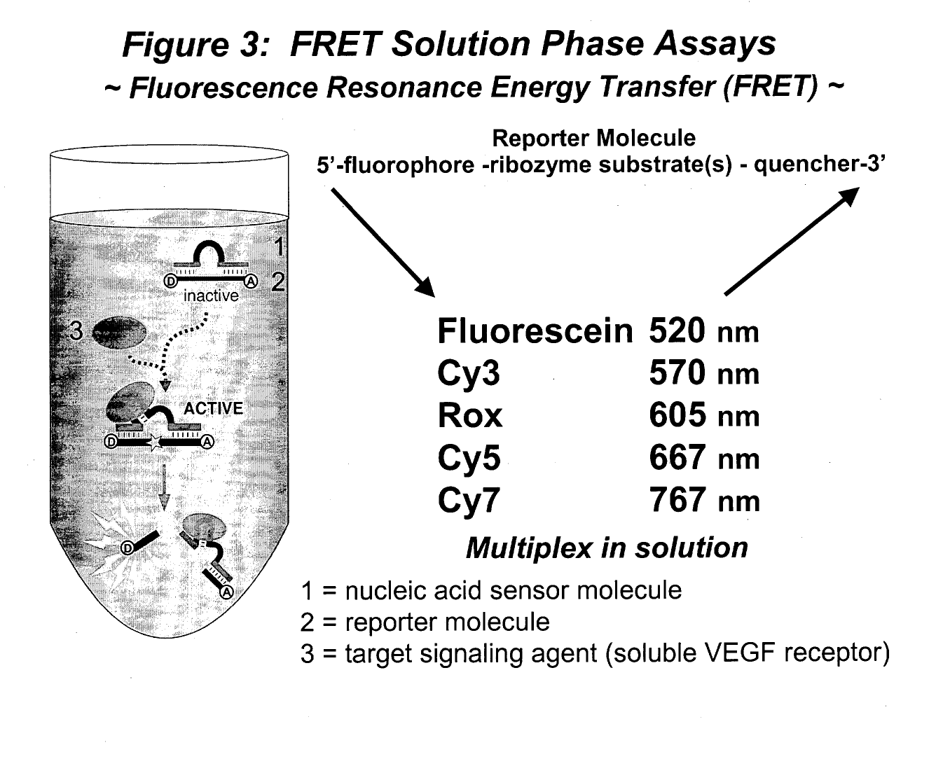

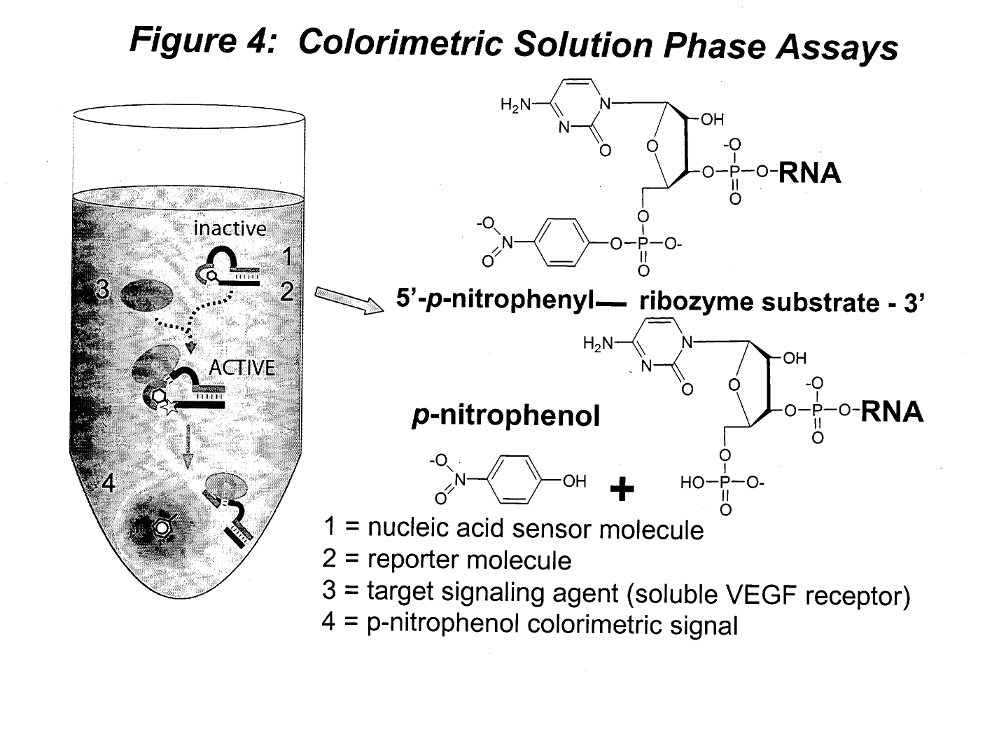

Nucleic acid sensor molecules are used to assay the presence of sVEGFR in biological samples. The nucleic acid sensor molecule is designed to detect the presence of sVEGFR using florescence resonance energy transfer (FRET) as shown in FIG. 3. Alternately, a colorimetric detection scheme is used as shown in FIG. 4.

In a non-limiting example, a tissue or blood sample is collected from a subject. This sample is then used as a component of a kit comprising a nucleic acid sensor molecule and reporter molecule of the invention, along with any other reagents such as buffers and excipients that are suited for the assay. The sample can be diluted with a buffer or used neat, and can optionally be partially purified or neutralized as the assay may require. The sample is then contacted with the nucleic acid sensor molecule under conditions suitable for detection of the target. In the presence of the target molecule, the nucleic acid sensor molecule catalyses a reaction that is detected by standard techniques, for example cleaving a nucleic acid substate comprising FRET moieties (FIG. 3) or by colorimetric assay (FIG. 4). The amount of signal is quantitated using a standard curve generated using known quantities of the effector molecule, for example an amount between 100 and 5,000 ug/l.

In one embodiment, the invention features a non-invasive diagnostic assay using human serum to detect sVEGFR levels and to establish levels of membrane bound VEGFR. In the practice of the invention, a sample of serum is collected from the subject to be tested. The collected sample is then subjected to diagnostic methods of the invention using ELISA, nucleic acid sensor molecules and/or aptamers which are capable of detecting sVEGFR if present in the sample.

The assay kit can comprise various devices, compartments, wells, channels or vessels to contain the various components of the kit and combine components when necessary. For example, the kit can comprise a device that allows loading of the sample followed by contact with the nucleic acid sensor and analytic read-out. Such a device can operate via liquid/liquid phase interaction or solid phase/liquid phase interaction using adsorption media or interactions on a surface. Devices for colorimetric and/or UV assay are also contemplated by the methods of the invention, as are automated processes of detection known in the art.

### Example 6

sVEGFR ELISA

ELISA is used for the quantitative determination of human sVEGFR concentrations in, for example, cell culture supernates, cell lysates, serum, plasma, saliva, and/or follicular fluid, and generally employs the quantitative sandwich enzyme immunoassay technique. In a non-limiting example, a monoclonal antibody specific for VEGFR is pre-coated onto a microplate. Standards and samples are pipetted in the wells and any VEGFR present is bound by the immobilized antibody. After washing away any unbound substances, an enzyme-linked polyclonal antibody speicific for VEGFR is added to the wells. Following a wash to remove any unbound antibody-enzyme reagent, a substrate solution is added to the wells and color develops in proportion to the amount of VEGFR obund in the intial step. The color development is stopped and the intensity of the color is measured (see for example Quantikine Human soluble VEGFR1 Immunoassay technical bulliten from R&D Systems Inc.).

### Example 7

Evaluation of Biomarkers in a Phase II Trial of Irinotecan/5-Fluorouracil/Leukovorin and ANGIOZYME in Metastatic Colorectal Cancer

Biological markers related to clinical outcome can provide insight into the development and use of targeted therapies. Five potential biomarkers were evaluated in an open-label Phase II trial of irinotecan/5-fluorouracil/leucovorin (IFL) and ANGIOZYME in 83 treatment-naive patients with metastatic colorectal cancer. ANGIOZYME is a stabilized ribozyme that targets the pre-mRNA of vascular endothelial growth factor receptor-1 (VEGFR-1) and could reduce both the cell surface receptor and its truncated, soluble form (sVEGFR-1). Patients received IFL and daily subcutaneous injections of 100 mg/m² ANGIOZYME. Plasma samples for 60 pts were obtained and analyzed for VEGF, sVEGFR-1, sVEGFR-2, sVCAM and sE-selectin at baseline and week 12 to determine the relationship between absolute level or changes in biomarkers and objective tumor response at week 12. Patients with detectable (>31.2 pg/mL) sVEGFR-1 at baseline (24/60) had fewer objective responses (9/29), a lower proportion of stable disease (7/19), and more progression (8/12) than patients with undetectable baseline levels of sVEGFR-1. In patients with detectable sVEGFR-1 at baseline, conversion to undetectable levels was associated with better clinical outcome at week 12 (CR+PR v SD v PD, p<0.01). In patients with undetectable sVEGFR-1 at baseline, conversion to detectable levels was not associated with clinical outcome (p=0.65). High pretreatment levels of sVCAM were associated with poorer outcome (p=0.03). In contrast, the level of sVEGFR-2 remained unchanged after treatment and was not related to clinical outcome. Objective response rate to IFL and ANGIOZYME was 43%, consistent with IFL treatment (Saltz, et al., 2000, NEJM, 343, 905-14). These data support further study of the prognostic value of sVEGFR-1 on clinical outcome in patients with metastatic colorectal cancer and whether therapies targeting VEGFR-1 offer an important advance in the treatment of this disease.

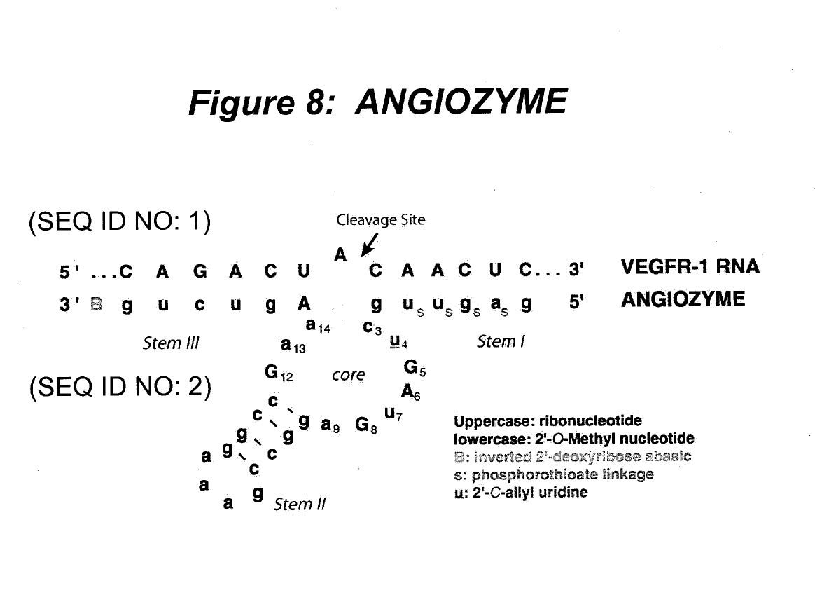

VEGF, VEGFR-1 and VEGFR-2 play important roles in the angiogenic process. ANGIOZYME, a stabilized ribozyme (FIG. 8), specifically targets the preRNA for VEGFR-1 and its alternatively-spliced, soluble form, sVEGFR-1. The consequence of ANGIOZYME-mediated decrease of this preRNA is expected to be concomitant with decreases in the membrane-bound and soluble forms of the receptor.

In preclinical studies in mice, ANGIOZYME inhibited the growth of the LLC-HM primary tumor in a dose-dependent manner, reduced the number of lung metastases and decreased the mean vascular density in tumor sections. ANGIOZYME also reduced the number of peritumoral vessels in a murine dermis model using HEY ovarian, SK-MEL-1 melanoma and ACHN renal carcinoma cell lines and was synergistic with interferon treatment. A reduction of metastasis in a human KM12 colorectal cancer xenograft and reduced metastasis and increase in survival in the murine mammary (4T1) model were also observed with ANGIOZYME treatment. ANGIOZYME was well absorbed by subcutaneous (SC) administration and well tolerated in 13-week toxicology studies at doses up to 300 mg/m²/d.

In several Phase I clinical trials, ANGIOZYME was well tolerated at doses up to 300 mg/m² administered IV or SC. In a Phase II monotherapy ANGIOZYME trial, 45 late-stage metastatic breast cancer patients who had failed multiple prior treatments received 100 mg/m² ANGIOZYME by daily SC injection. Due of the critical nature of the disease, most progressed and discontinued drug at the first evaluation (week 6). ANGIOZYME monotherapy was associated with a significant decrease in sVEGFR-1 by week 6. Here, applicant reports the results of the Phase II trial of ANGIOZYME in combination with IFL in previously untreated metastatic colorectal cancer patients. Clinical and biomarker results are summarized.

Phase II Clinical Study RPI0003

1. 83 patients with metastatic colorectal cancer enrolled

2. Daily SC ANGIOZYME (100 mg/m²) in combination with IFL

3. 24-week uncontrolled Phase II study (completed November 2002)

4. CT scans at 12 and 24 weeks

5. Research assays for potential biomarkers determined by validated ELISAs (R&D Systems) VEGF, sVEGFR-1, sVEGFR-2, sVCAM, and sE-selectin

1. Option for maintenance phase (RPI0102) with continued ANGIOZYME dosing upon completion of RPI0003

2. Primary endpoint: Response rate by RECIST (55% as evidence of ANGIOZYME effect)

3. Secondary endpoints: Stable disease at 24 weeks and time-to-progression (TTP)

Patients and Methods

Eligibility

1. Patients with measurable (RECIST criteria) and documented Stage IV metastatic adenocarcinoma of colon or rectum

2. No previous chemotherapy for metastatic colorectal cancer or disease free for more than 6 months from completion of adjuvant treatment

3. Karnofsky Performance Status (KPS)≧80%

4. Normal hepatic and renal function

5. No history of bleeding disorders

6. Written informed consent

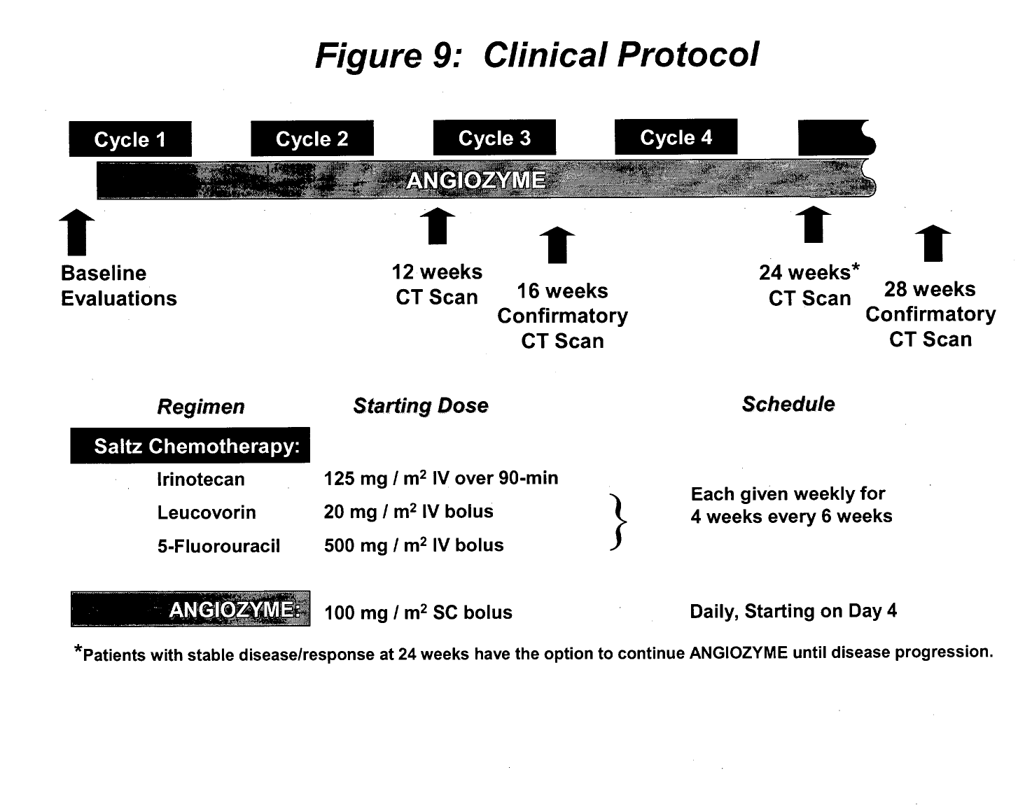

As shown in the protocol summarized in FIG. 9, chemotherapy (IFL) was started on study day 1 and continued as standard-of-care for up to 4 cycles. Beginning on study day 4, patients received daily doses of 100 mg/m² ANGIOZYME by subcutaneous injection. Unidimensional tumor measurements were obtained from CT scans after 12 and 24 weeks of treatment. Responses were then confirmed 4 weeks following initial response. ANGIOZYME treatment continued until disease progression. Patient demographics and baseline characteristics are given in Table 2. The disposition of patients with respect to treatment is given in Table 3.

Adverse event reporting for RPI0003 is ongoing for the 6 patients continuing to receive ANGIOZYME in the maintenance protocolis. ANGIOZYME was well tolerated. The most frequent adverse experiences for this and all other ANGIOZYME studies were injection site reactions (ISRs). One patient has discontinued RPI0003 due to an ISR.

The incidence of Serious Adverse Events (SAEs) is reported in Table 4. Of the MedDRA organ system classes most likely to be affected by antiangiogenic drugs, only the vascular complications (DVT/PE) may appear in excess in this study. In RPI0003 the 22 saxcular vascular SAEs in 19 of 83 (23%) patients may be higher than reported in trials of IFL alone. This observation was communicated to the FDA in September, 2002. Patients continue in the maintenance trial with all PEs and DVTs being classified as serious and ANGIOZYME-related to ensure complete reporting of these events.

The intent of the RPI0003 study (ANGIOZYME+IFL) was to compare response rates to an historical data set for a similar group of patients receiving IFL alone. The reference historical data set were the 231 patients randomized to the IFL arm of a Pharmacia study (Saltz, et al., 2000, NEJM, 343, 905-14). For a fair comparison, only patients in the Pharmacia study who actually received IFL, were of good performance status, and had CT scan results available at 12 or 24 weeks were included. In this analysis, response rates were similar (˜40%) for both studies. Median time to progression (TTP) was similar for both studies at 6.2 months for RPI0003 and >7.0 months for the IFL arm of the Pharmacia study (Table 5). ANGIOZYME was well tolerated as a daily SC injection given in combination with IFL. With the possible exception of increased vascular complications, ANGIOZYME added no toxicity to the chemotherapy.

RPI0003 Results: Analysis of sVEGFR-1 and Clinical Outcome

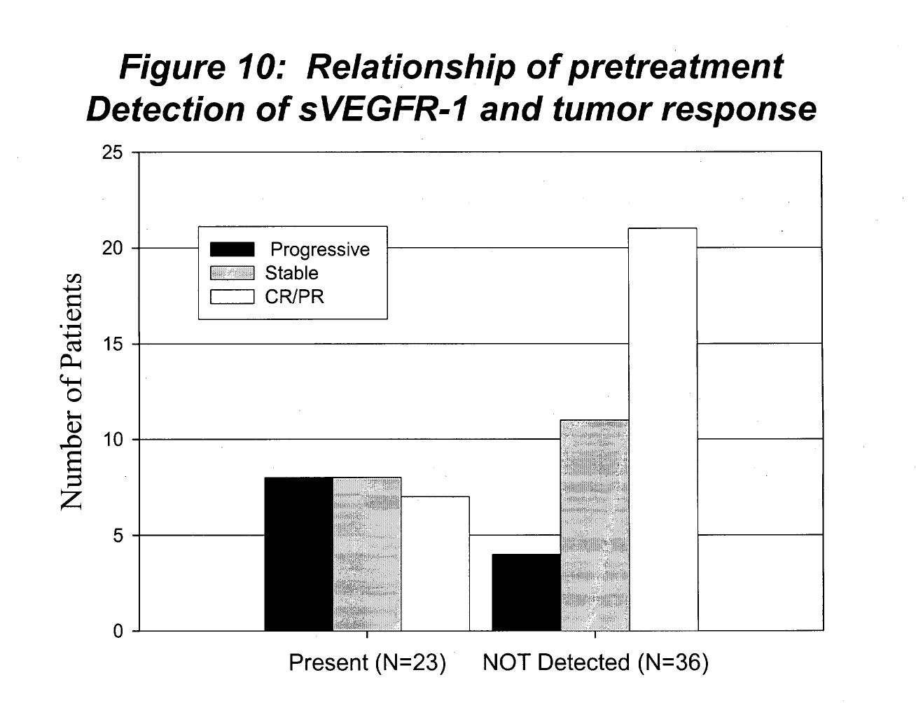

Blood samples from 59 eligible patients were analyzed for five potential biomarkers at baseline, 6 and/or 12 weeks (i.e. after at least one course of chemotherapy). Pre- and post-treatment levels of sVEGFR-1 were predictive of, and correlated with, clinical outcome in these patients (see FIGS. 10-13). Clinical outcome is measured by Best Overall Response (BOR) where objective response (CR/PR)>stable disease (SD)>progressive disease (PD).

Pretreatment levels of sVEGFR-1 Were Related to Clinical Outcome

sVEGFR-1 detected

detected in 23 patients (40%)

response and progression was similar among these 23 patients

sVEGFR-1 NOT detected

21 of 36 (58%) responded

4 of 36 (11%) progressed

Pretreatment absence of sVEGFR1 is related to improved tumor response, P=0.0170 (see FIG. 10)

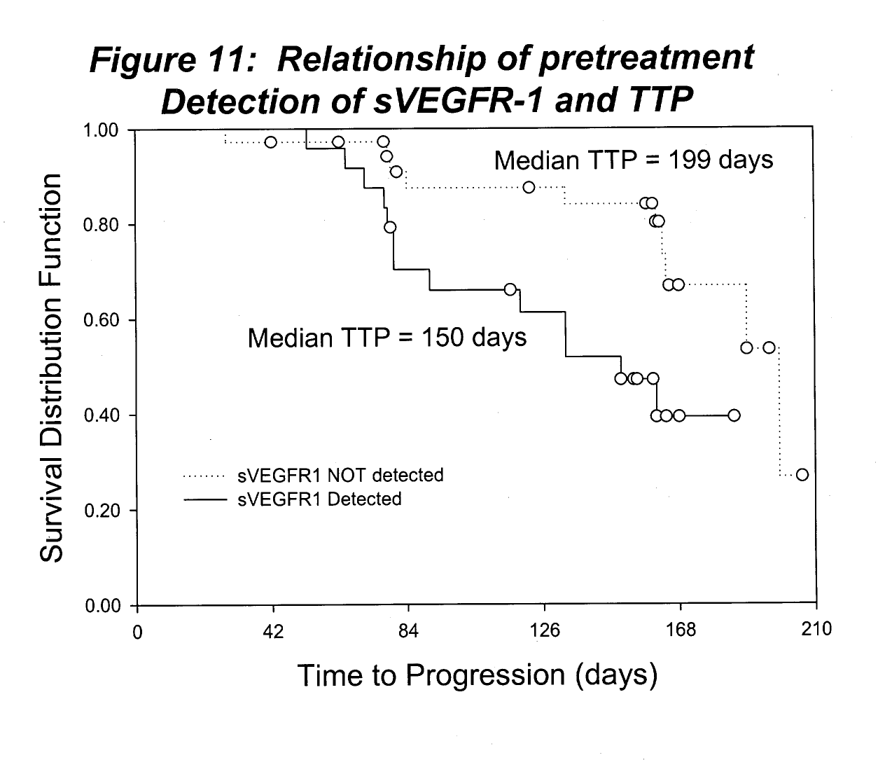

Median TTP was 7 weeks later among patients who presented with NO sVEBFR-1, Log-Rank p=0.0073 (see FIG. 11)

Changes in sVEGFR-1 are related to clinical outcome

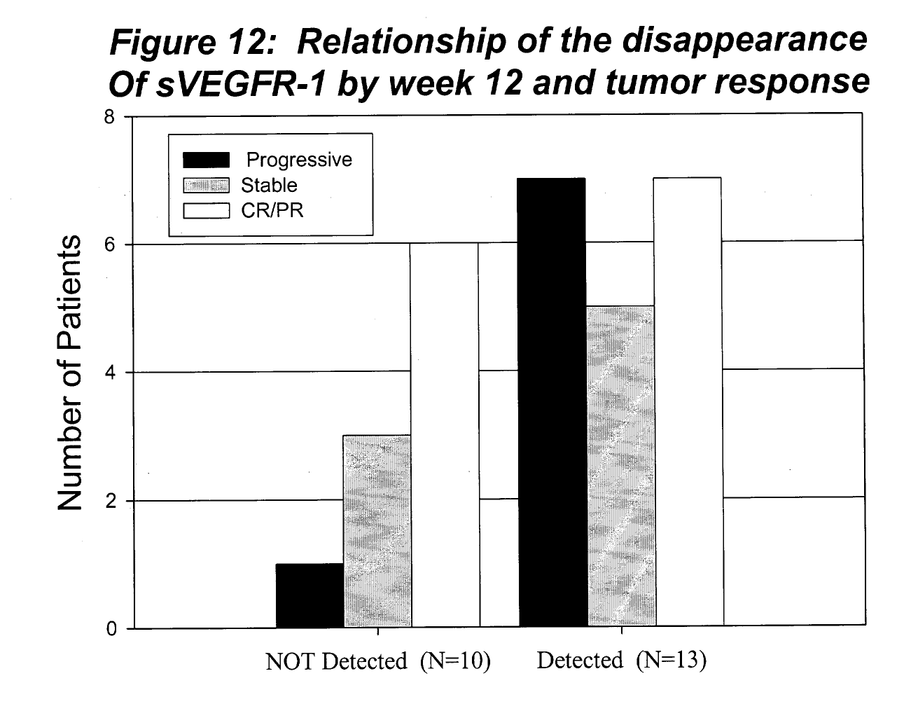

For the 23 patients with detectable sVEGFR-1 at pretreatment, disappearance of sVEGFR-1 is associated with a better clinical outcome (see FIG. 12).

sVEGFR1 was NOT detected at week 12 in 10 of 23 patients (43%)

6 of 10 responded

1 of 10 progressed

sVEGFR1 was detected at week 12 in 13 of 23 patients (57%)

1 of 13 responded

7 of 13 progressed

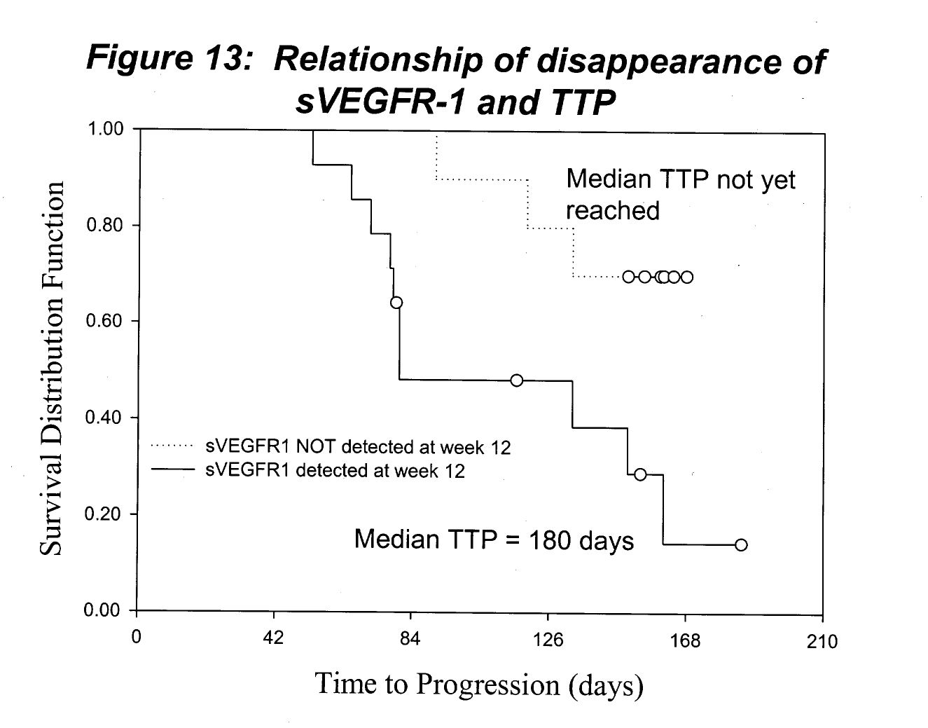

Disappearance of sVEGFR1 is associated with improved tumor response, P=0.0170. TTP was improved for patients where sVEGFR-1 had disappeared by week 12, Log-Rank (FIG. 13).

Other Potential Biomarkers

In a stepwise logistic regression to search for indicators of clinical outcome, VEGF, sVEGFR2 and sE-selectin did not appear to be related to clinical outcome. Higher levels of pretreatment sVCAM was associated with disease progression. Changes in sVCAM during treatment did not vary statistically by clinical outcome, but increases in sVCAM were highest for the patients who progressed. Importantly, the only biological indicator of clinical outcome was sVEGFR-1 at week 12 (P=0.001).

Summary and Conclusions

ANGIOZYME targets the preRNA of both the membrane-bound and soluble forms of VEGFR-1.

In RPI0001, ANGIOZYME monotherapy decreased sVEGFR-1 levels in the metastatic breast cancer setting, although there were no responses in this late-stage disease population.

In RPI0003, ANGIOZYME in combination with IFL in metastatic colorectal cancer resulted in two findings:

sVEGFR-1 NOT detected at pretreatment was associated with increased tumor response and longer disease progression TTP.

Decreases Disappearance of sVEGFR-1 by week 12 was associated with increased tumor response and longer TTP.

Since the colorectal trial was an uncontrolled combination trial, the effects of ANGIOZYME can not be separated from IFL or the combination. However, the specific action of ANGIOZYME on the VEGFR-1 preRNA target may result in the measurable decrease in sVEGFR-1 levels which are associated with improved clinical outcome.

Direct evidence that ANGIOZYME treatment decreases sVEGFR-1 and results in improved clinical outcome either as monotherapy or in combination with chemotherapy remains an objective of the ANGIOZYME development plan.

A randomized Phase II trial of chemotherapy±ANGIOZYME in a population of cancer patients with elevated pretreatment levels of sVEGFR-1 is a logical next development step.

The potential use of sVEGFR-1 as a predictive or therapeutic marker and of ANGIOZYME as a direct inhibitor of VEGFR-1 warrants further study.

### Example 8

Establishing Treatment in Cancer Subjects Using sVEGFR as a Prognostic Biomarker

Sera collected from subjects is assayed for sVEGFR and levels of sVEGFR are determined using an ELISA or other method of detection described herein or otherwise known in the art. Those patients demonstrating expression of sVEGFR (e.g., any level of sVEGFR greater than 0) are given a treatment that results in the down-regulation of VEGF or VEGF receptor expression, such as short interfering nucleic acid or siNA molecules (see for example McSwiggen et al., U.S. Ser. No. 60/399,348), enzymatic nucleic acid molecules (see for example Pavco et al., U.S. Ser. No. 09/870,161), antibodies (see for example Ooka et al, U.S. Pat. No. 5,730,977), proteins (see for example Zioncheck et al., U.S. Pat. No. 6,395,707), or small molecules targeting VEGF or VEGR receptors (e.g., VEGFR1 and/or VEGFR2).

### Example 9

Classification of Cancer Subjects Therapeutic Response Using sVEGFR as a Prognostic Biomarker, Use in Personalized Medicine

Although it is possible to determine the safety and efficacy of a drug for a group of people, it's difficult to determine whether a medicine is safe and effective for a specific person. Variables of different dosing requirements, the person's genotype, and the specific disease phenotype all contributing factors involved in effective treatment strategies. For example, an effective dose in one person may be ineffective in another person, a drug may work in one form of the disease and not work in another form of the disease, and a safe dose in one person may be unsafe in another. The use of screening cancer subjects for soluble VEGFR is useful in providing a personalized treatment approach to establish, for example, dosing, choice of therapy, and establishing criteria for effective prognosis or classification of the disease. Such personalized medicine approaches are expected to improve treatment efficacy, reduce healthcare cost, and provide better prognosis and monitoring of disease.

In a non-limiting example, sera collected from subjects is assayed for sVEGFR and levels of sVEGFR are determined prior to treatment, for example chemotherapy or treatment with nucleic acid therapeutics, such as siNA, aptamers, antisense, or enzymatic nucleic acid molecules, using an ELISA or other method of detection described herein or otherwise known in thee art. Following treatment over a certain time period (e.g. about 1-24 weeks etc.), levels of sVEGFR are determined from the analysis and are compared to a standard curve. Those patients with levels of sVEGFR that decrease over the time period (e.g., a decrease greater than 50%), are classified as having a good response and the treatment is continued. Those pateints with levels of sVEGFR that are essentially the same as prior to treatment are classified as having a neutral response (e.g. stable disease) and the treatement is continued or modified. Those patients with levels of sVEGFR that increase over the time period (e.g., an increase greater than 50%), are classified as having a poor response (disease progression) and the treatment regimen is reevaluated. As such, values of sVEGFR are used as a prognostic indicator of disease progression useful in evaluating and modifying treatment response.

### Example 10

Drug Discovery Using sVEGFR as a Biomarker of Efficacy

Levels of sVEGFR can be used as an endpoint in preclinical screening of therapeutic compositions useful in drug discovery and validation. Various animal models exist that can be used to screen therapeutic compositions for efficacy include the Lewis lung carcinoma and B-16 murine melanoma models that are both well accepted models of primary and metastatic cancer. These murine models are not dependent upon the use of immunodeficient mice, are relatively inexpensive, and minimize housing concerns. Both the Lewis lung and B-16 melanoma models involve subcutaneous implantation of approximately 10⁶ tumor cells from metastatically aggressive tumor cell lines (Lewis lung lines 3LL or D122, LLc-LN7; B-16-BL6 melanoma) in C57BL/6J mice. Alternatively, the Lewis lung model can be produced by the surgical implantation of tumor spheres (approximately 0.8 mm in diameter). Metastasis also can be modeled by injecting the tumor cells directly intravenously. In the Lewis lung model, microscopic metastases can be observed approximately 14 days following implantation with quantifiable macroscopic metastatic tumors developing within 21-25 days. The B-16 melanoma exhibits a similar time course with tumor neovascularization beginning 4 days following implantation. Since both primary and metastatic tumors exist in these models after 21-25 days in the same animal, multiple measurements can be taken as indices of efficacy. Primary tumor volume and growth latency as well as the number of micro- and macroscopic metastatic lung foci or number of animals exhibiting metastases can be quantitated. The percent increase in lifespan can also be measured. Thus, these models provide suitable primary efficacy assays for screening systemically administered therapeutic agents.

In the Lewis lung and B-16 melanoma models, systemic pharmacotherapy with a wide variety of agents usually begins 1-7 days following tumor implantation/inoculation with either continuous or multiple administration regimens. Concurrent pharmacokinetic studies can be performed to determine whether sufficient tissue levels of siRNA can be achieved for pharmacodynamic effect to be expected. Furthermore, primary tumors and secondary lung metastases can be removed and subjected to a variety of in vitro studies (i.e. target RNA reduction).

Soluble VEGFR protein levels can be measured clinically or experimentally as described herein. Furthermore sVEGFR mRNA levels are assessed by Northern analysis, RNase-protection, primer extension analysis and/or quantitative RT-PCR. Compositions that inhibit VEGFR and/or VEGF protein and/or RNA and therefore result in decreased levels of VEGF and/or VEGFR (e.g. VEGFR1 or VEGFR2) activity by more than 20% in vitro, as determined by sVEGFR levels, can be identified as candidates for clinical development.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. Thus, such additional embodiments are within the scope of the present invention and the following claims.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE 1
A. 2.5 μmol Synthesis Cycle ABI 394 Instrument

| | | | | Wait Time* | | Wait Time* 2′-O- | | | |
|---|---|---|---|---|---|---|---|---|---|
| Reagent | Equivalents | Amount | | DNA | | methyl | | Wait Time* RNA | |
| Phosphora | 6.5 | 163 | μL | 45 | sec | 2.5 | min | 7.5 | min |
| midites | | | | | | | | | |
| S-Ethyl | 23.8 | 238 | μL | 45 | sec | 2.5 | min | 7.5 | min |
| Tetrazole | | | | | | | | | |
| Acetic | 100 | 233 | μL | 5 | sec | 5 | sec | 5 | sec |
| Anhydride | | | | | | | | | |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | | | | |
|---|---|---|---|---|---|---|---|---|---|
| Phosphora | 15 | 31 | μL | 45 | sec | 233 | sec | 465 | sec |
| midites | | | | | | | | | |
| S-Ethyl | 38.7 | 31 | μL | 45 | sec | 233 | min | 465 | sec |
| Tetrazole | | | | | | | | | |
| Acetic | 655 | 124 | μL | 5 | sec | 5 | sec | 5 | sec |
| Anhydride | | | | | | | | | |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents: | Amount: | | | | | | | |
|---|---|---|---|---|---|---|---|---|---|
| | DNA/2′-O- | DNA/2′-O- | | Wait Time* | | Wait Time* | | Wait Time* | |
| Reagnet | methyl/Ribo | methyl/Ribo | | DNA | | 2′-O-methyl | | Ribo | |
| Phosphora | 22/33/66 | 40/60/120 | μL | 60 | sec | 180 | sec | 360 | sec |
| midites | | | | | | | | | |
| S-Ethyl | 70/105/210 | 40/60/120 | μL | 60 | sec | 180 | min | 360 | sec |
| Tetrazole | | | | | | | | | |
| Acetic | 265/265/265 | 50/50/50 | μL | 10 | sec | 10 | sec | 10 | sec |
| Anhydride | | | | | | | | | |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE 2
Patient Demographics and Baseline Characteristics

| | RPI0003 |
|---|---|
| | IFL + ANGIOZYME |
| Characteristic | (N = 83) |
| Sex, n/N (%) | |
| Female | 28/83 (34%) |
| Male | 55/83 (66%) |
| Age (years) | |
| Median | 57 |
| Range | 33-81 |
| <65 years, n (%) | 58/82 (70%) |
| ≧65 years, n (%) | 23/82 (28%) |
| ECOG Performance Status, | |
| n/N (%) | |
| 0 (KPS 90, 100) | 64/82 (77%) |
| 1 (KPS 80) | 17/82 (22%) |
| Number of Organs Involved, | |
| n/N (%) | |
| 1 | 33/82 (40%) |
| >1 | 49/82 (59%) |
| Liver Involvement, n/N (%) | |
| Yes | 64/83 (77%) |
| No | 19/83 (23%) |
| Prior Adjuvant Fluorouracil | |
| (5FU), n/N (%) | |
| Yes | 60/82 (27%) |
| No | 22/82 (73%) |
| Any Prior Radiotherapy, n/N (%) | 8/82 (10%) |
| Baseline Laboratory | |
| Abnormalities, n/N (%) | |
| White-cell count (>=8 × 10³/mm³) | 35/80 (44%) |
| Hemoglobin (<11 g/dL) | 11/80 (14%) |
| Lactate dehydrogenase (>ULN) | 37/77 (48%) |
| Total bilirubin (>ULN) | 0/75 (0%) |

TABLE 3
Patient Disposition

| | |
|---|---|
| Patients Enrolled | 83 |
| Ineligible | 2 |
| KPS = 70 | 1 |
| No measurable lesions | 1 |
| Eligible Patients | 81 |
| Completed the 24 week protocol | 39 |
| Withdrew during treatment: | 42 |
| Due to Disease Progression | 22 |
| Due to Adverse Event | 14 |
| Other | 6 |
| Enrolled in Maintenance Protocol | 34 |
| Remain on treatment (April 2003) | 6 |

TABLE 4
Incidence or Serious Adverse Events

| System Organ Class | Preferred Term | N Pts |
|---|---|---|
| Blood/Lymphatic | Febrile Neutropenia | 3 |
| | Neutropenia | 2 |
| | Anemia | 1 |
| Cardiac | Acute Myocardial | 1 |
| | Infarction | |
| | Atrial Fibrillation | 1 |
| Gastrointestinal | Abdominal Pain | 1 |
| | Diarrhea | 1 |
| | Gastrointestinal | 1 |
| | Hemorrhage | |
| | Intestinal Obstruction | 1 |
| | NOS | |
| Infections | Pneumonia | 1 |
| Metabolism and Nutritional | Dehydration | 1 |
| Musculoskeletal and | Vesicovaginal Fistula | 1 |
| Connective Tissue | | |
| Respiratory, Thoracic and | Alveolitis | 1 |
| Mediastinal | Dyspnea | 1 |
| | Respiratory Failure | 1 |
| Vascular | Arterial Thrombosis | 1 |
| | Deep Vein Thrombosis | 13 |
| | Superficial Vein | 1 |
| | Thrombosis | |
| | Pulmonary Embolism | 7 |

TABLE 5
Comparison of Response Rate and TTP between RPI0003 and
Pharmacia 0038

| | IFL | IFL + ANGIOZYME |
|---|---|---|
| | (N = 192) | (N = 81) |
| Best Overall Response (Week 24) | 37% | 41% |
| Unconfirmed at Week 12 | 48% | 44% |
| Confirmed at Week 12 | 35% | 33% |
| At least Stable at Week 24 | 80% | 76% |
| At least Stable at Week 12 | 83% | 78% |
| Discontinued by Week 12 | 17% | 22% |
| Due to Disease Progression | 12% | 16% |
| Due to Other Reasons | 5% | 6% |

What we claim is:

1. A method comprising:
(a) assaying a level of soluble VEGFR in a sample; and
(b) correlating said level with a level of membrane bound VEGFR in a subject from which said sample is derived.

2. The method of claim 1, wherein said sample is selected from the group consisting of blood, serum, urine, and tissue sample.

3. The method of claim 1, wherein said subject is a cancer subject.

4. The method of claim 3, wherein said cancer is colorectal cancer, breast cancer, or non-small cell lung cancer (NSLC).

5. The method of claim 1, wherein said assay comprises enzyme-linked immunosorbant assay (ELISA).

6. The method of claim 1, wherein said correlating comprises use of a standard curve of soluble VEGFR levels compared to membrane bound VEGFR levels.

7. A method for identifying cancer in a subject having elevated levels of membrane bound VEGFR, comprising:
(a) assaying a level of soluble VEGFR in a sample from the subject,
(b) determining a level of membrane bound VEGFR in said subject, and
(c) determining whether said subject has cancer based upon the level of soluble or membrane bound VEGFR in the subject.

8. The method of claim 7, wherein said sample is selected from the group consisting of blood, serum, urine, and tissue sample.

9. The method of claim 7, wherein said cancer is colorectal cancer, breast cancer, or non-small cell lung cancer (NSLC).

10. The method of claim 7, wherein said determining whether said subject has cancer based on the level of soluble or membrane bound VEGFR in the subject comprises use of a standard curve of soluble or membrane bound VEGFR levels compared to observed cancer phenotypes associated with soluble or membrane bound VEGFR levels.

11. A method for determining efficacy of a cancer treatment in a subject, comprising:
(a) assaying a level of soluble VEGFR in a sample from said subject prior to said cancer treatment,
(b) assaying a level of soluble VEGFR in a sample from said subject during or after said cancer treatment; and
(c) determining efficacy of said treatment by comparing the level of soluble VEGFR in a sample from said subject prior to said cancer treatment to the level of soluble VEGFR in a sample from said subject during or after said cancer treatment.

12. The method of claim 11, wherein said cancer treatment comprises treatment with a siNA molecule.

13. The method of claim 11, wherein said sample is selected from the group consisting of blood, serum, urine, and tissue.

14. The method of claim 11, wherein said cancer is colorectal cancer, breast cancer, or non-small cell lung cancer (NSLC).

15. The method of claim 11, wherein a level of soluble VEGFR that is lower in (b) compared to (a) indicates efficacy for a cancer treatment.

16. A method for predicting prognostic outcome of a cancer subject, comprising:
(a) assaying a level of soluble VEGFR in a sample from said subject, and
(b) using the level of soluble VEGFR in the subject under conditions suitable to predict prognostic outcome of said subject.

17. A method for predicting prognostic outcome of a subject undergoing cancer treatment, comprising:
(a) assaying a level of soluble VEGFR in a sample from said subject prior to said cancer treatment,
(b) assaying a level of soluble VEGFR in a sample from said subject during or after cancer treatment, and
(c) predicting prognostic outcome of said subject undergoing said cancer treatment by comparing the level of soluble VEGFR in a sample from said subject prior to said cancer treatment to the level of soluble VEGFR in a sample from said subject during or after cancer treatment.

18. The method of claim 17, wherein said cancer treatment comprises treatment With a siNA molecule.

19. The method of claim 17, wherein said sample is selected from the group consisting of blood, serum, urine, and tissue sample.

20. The method of claim 17, wherein said cancer is colorectal cancer, breast cancer, or non-small cell lung cancer (NSLC).

21. The method of claim 17, wherein a level of soluble VEGFR that is lower in (b) compared to (a) indicates efficacy for a treatment.


SEQUENCE LISTING (3 sequences)

SEQ ID NO: 1 - RNA, 13 nt, Artificial
  VEGFR-1 RNA
cagacuacaacuc

SEQ ID NO: 2 - RNA, 34 nt, Artificial
  Angiozyme
gaguugcugaugaggccgaaaggccgaaagucug

SEQ ID NO: 3 - RNA, 83 nt, Artificial
  Nucleic Acid Sensor
ggauaauagccguagguugcgaaagcgacccugaugagnnnnnnnnnnnnnnnnnnnnnnnnncgaaacgguagcgagagcuc